(19) United States
(12) Patent Application Publication
(10) Pub. No.: US 2019/0159963 A1
(43) Pub. Date: May 30, 2019

(54) SPLIT PHASE VENTILATION FOR CPR AND METHODS

(71) Applicant: Keith G. Lurie, Minneapolis, MN (US)

(72) Inventor: Keith G. Lurie, Minneapolis, MN (US)

(21) Appl. No.: 16/201,339

(22) Filed: Nov. 27, 2018

Related U.S. Application Data
(60) Provisional application No. 62/591,029, filed on Nov. 27, 2017.
(60) Provisional application No. 62/599,465, filed on Dec. 15, 2017.

(51) Int. Cl. A61H 31/00; A61M 16/00

(52) U.S. Cl. CPC A61H 31/008; A61H 31/005; A61M 16/024; A61H 31/006; A61H 2201/0192; A61H 2201/107; A61M 2230/40; A61M 2205/3344; A61H 2203/0456

(57) ABSTRACT

A method for ventilating a patient during CPR including repeatedly compressing the patient's chest and delivering a positive pressure ventilation to the patient primarily during a decompression phase of the repeated compressions of the patient's chest.

### CROSS-REFERENCES TO RELATED APPLICATION

This application claims the benefit of U.S. Provisional Application. No. 62/591,029 filed Nov. 27, 2017, and titled “SPLIT PHASE VENTILATION FOR CPR AND METHODS,” and this application claims the benefit of U.S. Provisional Application No. 62/599,465, filed Dec. 15, 2017, and titled “SPLIT PHASE VENTILATION FOR CPR AND METHODS,” which are hereby incorporated herein in their entireties by reference for all purposes.

### BACKGROUND OF THE INVENTION

Closed chest CPR (CCC) is inherently inefficient, but in the absence of a beating heart it can be lifesaving. Nonetheless, overall US national survival rates for out of hospital cardiac arrest (OHCA) with favorable neurological function remains poor, about 7-8%, despite the practice of CCC for >50 years. No EMS system has been able to achieve values overall survival rates with favorable brain function of >25% for all presenting patients. Progress is desperately needed.

CCC is insufficient for multiple reasons, including the fact that the delivery of each positive pressure breath results in an increase in intrathoracic pressure. This results in an immediate decrease in venous blood flow back to the heart and reduced refilling of the heart after each compression, a rise in intracranial pressure, a decrease in cerebral perfusion, and potential lung injury and trauma due to the competing physical forces of forcibly inflating the lungs while simultaneously compressing the chest and the lungs within the chest cavity.

### BRIEF SUMMARY OF THE INVENTION

In one embodiment, a method for ventilating a patient during CPR is provided. The method may include repeatedly compressing the patient's chest resulting in repeating compression and decompression phases of the patient's chest and delivering a positive pressure ventilation to the patient during only multiple successive decompression phases of the repeated compressions of the patient's chest. In some embodiments, the method may also include sensing one or both of a compression phase and the decompression phase of the repeated compressions of the patient's chest. Delivering the positive pressure ventilation may be based on the sensing. In some embodiments, sensing one or both of a compression phase and the decompression phase of the repeated compressions of the patient's chest may include using at least one of a sensor attached to the patient, an airway adjunct attached to the patient, or a compression sensor in a chest compression device that is interfaced with the patient. A sensed signal from at least one of the sensor, the airway adjunct, or the compression sensor may trigger the positive pressure ventilation during the decompression phase. In some embodiments, the method may further include actively decompressing the patient's chest between each compression of the patient's chest and/or elevating the individual's heart, shoulders, and head relative to horizontal while repeatedly compressing the patient's chest. In some embodiments, elevating the individual's heart, shoulders, and head is performed at a rate of between about 0.25°/second and about 40°/minute.

In another embodiment a method for ventilating a patient during CPR includes repeatedly compressing the patient's chest resulting in repeating compression and decompression phases of the patient's chest, detecting a decompression phase of the patient's chest, and initiating delivery of a positive pressure ventilation to the patient based on the detected decompression phase. The positive pressure ventilation may be delivered at least primarily during the detected decompression phase. In some embodiments, the positive pressure ventilation may be delivered to the patient only during the multiple successive decompression phases. In some embodiiments, the method may also include detecting a compression phase of the patient's chest and ceasing the delivery of the positive pressure ventilation upon the detection of the compression phase. In some embodiments, the method may further include detecting a subsequent decompression phase of the patient's chest and resuming the delivery of the positive pressure ventilation to the patient based on the detected subsequent decompression phase. In some embodiments, the method may include elevating the individual's heart, shoulders, and head relative to horizontal while repeatedly compressing the patient's chest. The individual's heart, shoulders, and head may be elevated in a controlled manner over a period of time between about 20 seconds and 10 minutes.

In another embodiment, a system for delivering split-phase positive pressure ventilations for use in cardiopulmonary resuscitation (CPR) is provided. The system may include a positive pressure delivery device and at least one sensor that is configured to be coupled to one or both of a patient or a device that interacts with a patient during CPR and detect a decompression phase of CPR. The system may also include a controller system that is coupled with the positive pressure delivery device and in communication with the at least one sensor. The controller system may be configured to cause the positive pressure delivery device to deliver positive pressure ventilation primarily during the decompression phase of CPR over at least two decompression cycles. In some embodiments, the at least one sensor is further configured to detect a compression phase of the patient's chest and the controller system is further configured to cease the delivery of the positive pressure ventilation base on the detection of the compression phase. In some embodiments, the at least one sensor includes an intrathoracic pressure sensor that is configured to detect the compression phase when an intrathoracic pressure of the individual exceeds a predetermined threshold. In some embodiments, the predetermined threshold is about 10 mmHg.

In some embodiments, the system may include an elevation device having a base and an upper support coupled with the base. The upper support may be configured to elevate an individual's heart, shoulders, and head relative to horizontal. The upper support may be moveable between a first elevated position and a second elevated position. The second elevated position has a level of elevation relative to the horizontal which is greater than that of the first elevated position. The elevation device may also include an adjustment mechanism coupled with the upper support that is configured to adjust a level of elevation of the upper support and a chest compression device for performing chest compressions coupled with one or both of the base and the upper support. In some embodiments, the chest compression device is further configured to actively decompress the individual's chest between each chest compression. In some embodiments, the elevation device further includes an additional controller system that is coupled with the adjustment mechanism and the chest compression device. The additional controller system may be configured to actuate the chest compression device for a period of time with the upper support in the first elevated position, subsequently move the upper support from the first elevated position to the second elevated position whilst the chest compression device remains actuated, and actuate the chest compression device with the upper support in the second elevated position. In some embodiments, the controller system and the additional controller system are the same.

### BRIEF DESCRIPTION OF THE DRAWINGS

FIG. 1A is a schematic of a patient receiving CPR in a supine configuration according to embodiments.

FIG. 1B is a schematic of a patient receiving CPR in a head and thorax up configuration according to embodiments.

FIG. 2A is a schematic showing a configuration of HUP CPR according to embodiments.

FIG. 2B is a schematic showing a configuration of HUP CPR according to embodiments.

FIG. 2C is a schematic showing a configuration of HUP CPR according to embodiments.

FIG. 3A depicts an elevation device in a lowered position according to embodiments.

FIG. 3B depicts the elevation device of FIG. 3A in an elevated position.

FIG. 3C depicts a locking handle of the elevation device of FIG. 3A.

FIG. 3D depicts the locking handle of the elevation device of FIG. 3A.

FIG. 3E depict a linear actuator of the elevation device of FIG. 3A in an elevated position.

FIG. 3F depict the linear actuator of the elevation device of FIG. 3A in a lowered position.

FIG. 3G depicts the elevation device of FIG. 3A in a lowered position.

FIG. 3H depicts the elevation device of FIG. 3A in an elevated position.

FIG. 3I depicts a latch of the elevation device of FIG. 3A.

FIG. 3J depicts the latch of the elevation device of FIG. 3A.

FIG. 3K depicts a release knob of the elevation device of FIG. 3A.

FIG. 3L depicts a release cable of the elevation device of FIG. 3A.

FIG. 4 depicts an elevation device with a chest compression device according to embodiments.

FIG. 5 is a flowchart of a process for ventilating a patient during CPR according to embodiments.

FIG. 6 depicts a chart demonstrating examples of the physiological effects of split phase ventilation according to embodiments.

### DETAILED DESCRIPTION OF THE INVENTION

The present invention reduces the potential adverse effects of positive pressure ventilation during CCC by providing a means to time the delivery of a breath over a 1 to 2 second period of time during the chest compression/decompression cycle and to split the delivery of the breath up based upon the timing of the changes in intrathoracic pressure during the compression and decompression phase of the CCC cycle and some portion or no portion of the compression phase. More specifically, this invention provides a means to deliver a breath selectively during all or part of the decompression phase of the CCC compression/decompression cycle. This invention results in lower mean and peak intrathoracic pressure values and thereby improves respiration and circulation of blood to the brain, heart, and other parts of the body.

During CPR, circulation of blood to the brain and heart is severely compromised. Manual and current automated closed chest CPR (CCC) techniques provide less than 30% of normal blood flow to the brain and the heart. Over time this is further reduced. When compressions are stopped to provide a positive pressure ventilation, blood flow to the heart and brain is further compromised since there is no circulation without chest compressions during CPR.

Ventilation during CPR is important, but not as important as maintaining circulation. As such, in recent years the American Heart Association has shifted the focus to starting chest compressions first and reducing the overall ventilation rate. The recommended frequency of ventilations has decreased over the past 50 years as the recommended compression rate has increased. For example, the American Heart Association used to recommend five compressions and then one breath, followed by a switch to 15 compressions and two breaths for basic life support providers, before most recently switching to a recommendation of 30 compressions and two breaths.

When a patient with a stable blood pressure and heartbeat is mechanically ventilated, a positive pressure breath can be delivered over a period of time, usually 1-2 sec. This usually results in a rise in intrathoracic pressure of about 10-20 mmHg with minimal physiological benefits if the patient's intravascular volume status is within normal limits. When a patient with a stable heartbeat but with low blood pressure (due to hemorrhage, for example) is mechanically ventilated, each positive pressure breath results in a transient but immediate decrease in arterial blood pressure and blood flow to the heart and brain. This occurs since the positive pressure in the thorax from each breath reduces the return of venous blood back into the right heart and elevates intracranial pressure. This physiology is well known and has resulted in recommendations to avoid excessive ventilation rates and ventilation tidal volumes in patients with low blood pressure.

However, when a patient is in cardiac arrest the heart stops, so the external chest compressions provide the primary means for promoting forward blood flow. The physiological interactions between positive pressure ventilations and intrathoracic pressures are fundamentally different from the physiology of a living person. With each compression, CCC increases the pressures within the thorax to upwards of 100 mmHg. The air within the lungs helps to cushion the rise in pressures with the lungs themselves, especially as air is pushed out of the lungs with each chest compression. In the absence of synchrony between chest compressions, each time a positive pressure breath is delivered and the chest is compressed during CPR the intrathoracic pressure rises. This increase varies from compression to compression and positive pressure breath to breath. At times the maximum intrathoracic pressure or peak inspiratory pressures can exceed 45 mmHg. Moreover, it is difficult if not impossible to completely deliver the desired tidal volume within a <0.5 to 1.0 sec as recommended in Heart Association guidelines to avoid high peak inspiratory pressures. This is because the external chest compression increases the pressure inside the thorax to such high levels with each chest compression that it is difficult with conventional ventilation techniques to overcome the high intrathoracic pressures.

When a pressure cycled ventilator is used during CPR, only part of the breath is delivered to the patient. Once the pressure limit is reached, the ventilator no longer delivers a breath. This results in under ventilation or hypoventilation.

When a volume cycled ventilator is used during CPR, it will deliver the breath regardless of the pressure in the thorax. This can result in dangerously high intrathoracic pressures. If a combination device is used to deliver a breath, this can have untoward consequences, as it can result in hypoventilation and excessively high and dangerous pressures.

The present invention provides a new technique to deliver a positive pressure breath over a 1 to 2 second period of time during CPR and to split the delivery of the breath based upon the compression and decompression cycle as well as the relative pressures in the thorax during the compression and decompression cycle. Such split-phase ventilation techniques may be utilized both in conventional, supine CPR and/or in head up (HUP) CPR. When the positive pressure breath is delivered and a sensor detects a rise in intrathoracic pressure from chest compressions, (and/or using timing mechanisms that connect the compression source and the ventilation source to gate the ventilation timing), then the delivery of the breath is interrupted until the start of the next decompression phase cycle, at which point the ventilator completes the delivery of the breath. In other words, the compression device and ventilation device work synchronously so that the delivery of the breath is split at least once, if not twice, and synchronized with the decompression phase of the compressor. Thus, the delivery of the breath may initially increase intrathoracic pressure to about 10-20 cm of water, stop for less than one second during the compression phase, and then resume at the start of the next decompression phase. In this way a positive pressure breath can be delivered periodically (4-16 times per min) during CPR without limiting the tidal volume delivered, causing excessively high peak inspiratory pressures, or stopping chest compressions for the delivery of a breath.

In another embodiment, an impedance threshold device is used to restrict the inflow of respiratory gases into the lungs except when a positive pressure breath is delivered. Sensors in the impedance threshold device are used to determine the start of the chest compression and/or duty cycle and can also help to regulate the split phase ventilation either directly or by feedback to the ventilator. The invention may utilize any of the threshold valves or procedures using such valves that are described in U.S. Pat. Nos. 5,551,420; 5,692,498; 5,730,122; 6,029,667; 6,062,219; 6,810,257; 6,234,916; 6,224,562; 6,526,973; 6,604,523; 6,986,349; and 7,204,251, the complete disclosures of which are herein incorporated by reference.

At the current time the American Heart Association recommends the delivery of one breath in an asynchronous manner every 10 compressions. Unlike normal breathing, which includes the generation of negative intrathoracic pressure to draw air into the lungs, during standard CPR a positive pressure breath is delivered either by mouth-to-mouth ventilation or with a resuscitator bag and an airway adjunct. Each time a positive pressure breath is delivered to a patient who has low blood circulation and blood pressure (which includes effectively all patients in cardiac arrest), blood flow back to the heart, termed venous return, is diminished. In addition, intracranial pressure is increased due to the immediate transmission of a positive pressure from the thorax to the brain. The correct balance between delivering a positive pressure breath, as recommended by the ‘American Heart Association,’ with a tidal volume of about 500-600 mL (8-10 ml/kg) and a duration of about one second and the need to provide circulation is nearly impossible to find.

Moreover, there are multiple issues with the delivery of positive pressure ventilation during CPR. When a manual resuscitator bag is used, rescuers often to ventilate too frequently, as reported by the inventor nearly 20 years ago as discussed in Lurie, Keith G. (2004) “Death By Hyperventilation: A Common and Life-Threatening Problem During Cardiopulmonary Resuscitation,” Crit Care Med. 2004 September; 32(9 Suppl):S345-51, Aufderheide TP, the entire contents of which is hereby incorporated by reference. The high ventilation rates are associated with almost continuously high intrathoracic pressure which prevents venous return and refilling of the left ventricle. The excessive ventilations cause a decrease in forward flow out of the heart, or cardiac output and a harmful increase in intracranial pressure. The higher the intracranial pressure, the greater the resistance to forward blood flow to the brain. In addition, due to the timing of the chest compressions, which should be delivered at a rate of between 100 and 110 per minute, and the asynchronous breath, which should be delivered between 8 to 12 times per minute, it is virtually impossible to deliver the positive pressure breath on the upstroke of the chest compression decompression cycle. As a consequence, frequently when a positive pressure breath is delivered, there is already a high intrathoracic pressure from the chest compression that is being delivered concurrently. The result is a dangerously high generation of intrathoracic pressure and at times intracranial pressure as well as inadequate delivery of the title volume of oxygen.

Historically some inventors created devices to deliver positive pressure breaths only during the compression phase of CPR. This has resulted in higher intrathoracic pressures and higher arterial pressures. Few, if any, have looked at blood flow to the heart and brain with this kind of intervention. It is known that high intrathoracic pressures resulting from the delivery of excessive positive pressure ventilation can cause a barotrauma to the lungs but moreover can increase intracranial pressure to dangerous levels. Given the mismatch between the recommendation for the delivery of a breath over one second and the compression decompression phase oscillations of 300 ms each when CPR is delivered at 100 times per minute, less than half of the time a positive pressure breath is delivered during the decompression phase.

The delivery of a positive pressure breath while simultaneously compressing the chest is ineffective from the perspective of ventilation and is dangerous from the perspective of generation of high intra-thoracic pressure and peak inspiratory pressure. This invention remedies this potentially ineffective and harmful current ventilation strategy with a new method and device to ventilate during CPR.

The new method and devices are based upon the concept of delivering an effective tidal volume to the lungs selectively during the decompression phase of CPR. As such, the positive pressure ventilation will need to be split between one or more chest compression phases. In some cases the positive pressure breath delivered during the decompression phase may extend into a portion of the compression phase. This may depend upon a sensed physiological measurement, for example, the intrathoracic pressure or airway pressure. The split phase ventilation method includes a means to sense the compression and decompression cycles and delivery of a positive pressure breath that is controlled by the amount of volume delivered or the peak pressures that are achieved or both combined. There are multiple ways that the chest compression and decompression cycle can be sensed and some of these include a pressure or force sensor attached to the chest, a change in electrical impedance across the chest, a pressure or force sensor attached to some part of the patient's body or to the machine that is delivering the CPR or to the bed or platform that the patient is resting on, and/or combinations thereof. In addition, chest compression and decompression cycles could be sensed by force, flow, and/or pressure transducers in a device attached to the airway such as an endotracheal tube or impedance threshold device, or an active intrathoracic pressure regulator device, the latter which simultaneously is used to regulate airflow in and out of the lungs and/or intrathoracic pressures.

Information from the sensed compression and decompression cycle is then used to regulate a timer either on a manual or automated positive pressure generating machine. In this manner, during each decompression phase, a positive pressure breath can be delivered over a duration of time sufficient for the delivery of the title volume. However, as soon as the compression phase is sensed the positive pressure ventilation delivery is halted until the start of the next decompression phase. The duration of the breath can be regulated depending on the physiological needs of the patient but in general the breath would be delivered over about 0.75 to 2.0 seconds with part of the breath delivered before each compression and the other part of the breath delivered after the compression phase.

The amount of oxygen a patient needs will depend on their metabolic state. Typically during the low blood flow state of cardiac arrest, even with reasonable blood flow during CPR, oxygen demand is low. That is why only 8 to 12 breaths are recommended with standard CPR.

Circulation during CPR can be increased using active compression decompression CPR and the impedance threshold device or other forms of intra-thoracic pressure regulation. In addition, circulation can be increased, especially to the brain, when the head and thorax are elevated during CPR. Systems and method for the performance of CPR with the head and thorax elevated are described in U.S. Patent Publication No. 2015/0231026, U.S. Patent Publication No. 2015/0231027, U.S. Patent Publication No. 2015/0231027, U.S. Pat. Nos. 9,707,152, 9,707,152, 9,801,782, U.S. Patent Publication No. 2016/0338904, U.S. Patent Publication No. 2017/0119622, and U.S. Patent Publication No.-2017/0258677, the complete disclosures of which are herein incorporated by reference.

The invention anticipates the multiple different ways one can perform CPR and the resultant differences in the duration of each breath and the tidal volume of oxygen and other respiratory gases needed for delivery during CPR. In some cases the device will be part of a portable ventilator system and it will have a CPR mode that can be switched to a non-CPR mode. In some cases the sensors will be incorporated into an impedance threshold device that is capable of being linked electronically, either by Bluetooth or hardwire, to the positive pressure ventilation source. In some cases physiologic algorithms, including computerized Machine learning and artificial intelligence, will be used to guide the duration and delivery of the breath during the decompression phase of CPR. The duration of the breath and the tidal volume and the speed at which it is delivered could potentially vary from one breath to the next depending upon the machine learning algorithm feedback loop that is intended to maximize blood flow to one or more vital organs. Further, the actual duty cycle and excursion distance (depth and amount of active recoil) of the compression decompression cycle may be varied, which may allow the ventilations and circulation to be further optimized. For example, the decompression phase of the CPR cycle may be increased from 50% to 60-70% of the entire decompression cycle. In such circumstances, the new invention would continue to sense the compression and decompression cycles and deliver the breath accordingly, during the decompression phase only. In some embodiments, the sensing of the compression and/or decompression cycle may be done for example, using external visualization, using light, laser, infrared, external sensors that are not attached to the patient, the ventilation device, or the compression device, and/or combinations thereof. It will be appreciated that other techniques for detecting the compression and/or decompression phases of CPR and these techniques may be used in accordance with the present invention. Still in another example, an intrathoracic pressure regulator, would alter the speed at which a positive pressure breath could be delivered as the generation of negative intrathoracic pressure will accelerate air into the lungs from each positive pressure breath. Techniques as well as equipment and devices for intrathoracic pressure regulation are also described in U.S. patent application Ser. Nos. 11/034,996 and 10/796,875, and also U.S. Pat. Nos. 5,730,122; 6,029,667; 7,082,945; 7,410,649; 7,195,012; and 7,195,013, the complete disclosures of which are herein incorporated by reference. In yet another embodiment, a positive pressure breath would only be delivered as long as the sensed intrathoracic pressure was always less than a certain value, for example 20 mmHg. In another embodiment, intrathoracic pressure would be sensed and the changes in intrathoracic pressure would be used as a surrogate for compression and decompression cycle timing. An intrathoracic pressure sensor may be incorporated into, for example, an impedance threshold device, which may be used to time the positive pressure breath during the decompression phases of CPR.

It is anticipated that many of the commercially available ventilators could have this additional CPR mode easily added to them by minimal electronic circuitry changes.

In a more sophisticated system, the ventilator may be configured to sense that the patient is trying to take a breath, a so-called agonal respiration or gasp. In some embodiments the delivery of the positive pressure breath would still be limited to only the period of time when the chest wall is recoil occurs either passively or actively during CPR. It is further noted that the ventilator could be adjusted so that the timing and amount of the breath, in terms of the speed and waveform of volume delivery, could also be altered based upon the physiological needs of the patient. Moreover, this may vary when the patient is flat versus with head and thorax elevation and it may also very depending on the method of a CPR used as well as the method used to regulate intrathoracic pressure. In addition, in some cases the angle between the chest compressor and the chest may vary between 70 degrees and 110 degrees. In such cases, the split phase ventilator may also vary such that the positive pressure ventilations are delivered during the chest wall recoil phase and a portion but not all of the compression phase. In some cases it may be advantageous to provide split phase ventilation but instead of generating a negative intrathoracic pressure during the decompression phase with the addition of an intrathoracic pressure regulator device the intrathoracic pressure may be slightly positive (1-5 cm H2O) during portions or all of the decompression phase.

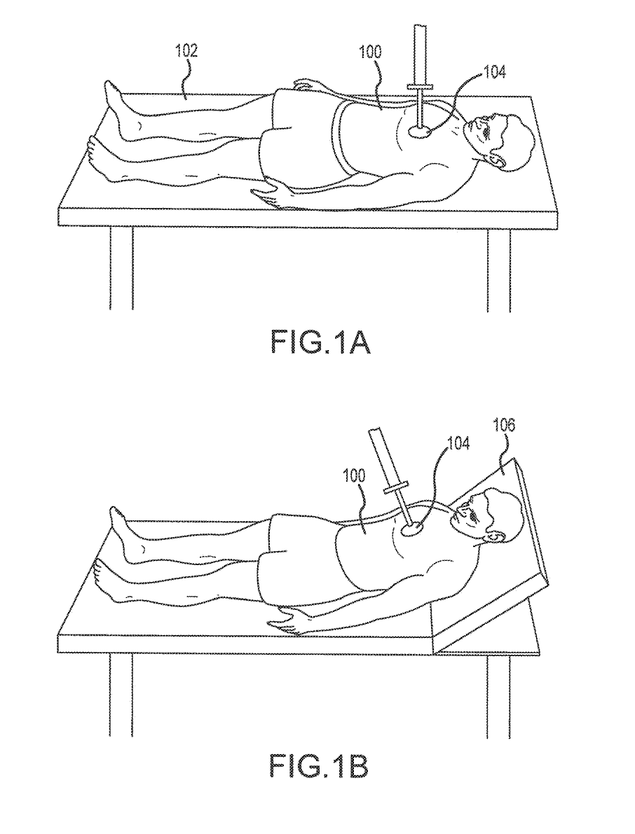

Turning now to FIG. 1A, a demonstration of the standard supine (SUP) CPR technique is shown. Here, a patient 100 is positioned horizontally on a flat or substantially flat surface 102 while CPR is performed. CPR may be performed by hand and/or with the use of an automated CPR device and/or ACD+CPR device 104. In contrast, a HUP CPR technique is shown in FIG. 1B. Here, the patient 100 has his head and thorax elevated above the rest of his body, notably the lower body. The elevation may be provided by one or more wedges or angled surfaces 106 placed under the patient's head and/or thorax, which support the upper body of the patient 100 in a position where both the head and thorax are elevated, with the head being elevated above the thorax. HUP CPR may be performed with conventional standard CPR alone, with ACD alone, with the ITD alone, with the ITD in combination with conventional standard CPR alone, and/or with ACD+ITD together. Such methods regulate and better control intrathoracic pressure, causing a greater negative intrathoracic pressure during CPR when compared with conventional manual CPR. In some embodiments, HUP CPR may also be performed in conjunction with extracorporeal membrane oxygenation (ECMO). HUP CPR can also be performed with a number of different automated CPR devices, including those that compress the chest and allow for passive or active recoil, (e.g., ACD CPR with a ResQPump or LUCAS device) and those that also circumferentially compress the chest, with or without active chest recoil. HUP CPR can also be performed with a load-distributing band CPR (e.g. AutoPulse) with or without an element of sternal compression and/or decompression, circumferential chest compression, external compression with an intra-aortic balloon (e.g. REBOA), any of the aforementioned methods of CPR plus a means to additionally regulate intrathoracic pressure, such as an impedance threshold device e.g. ResQPOD), and the like.

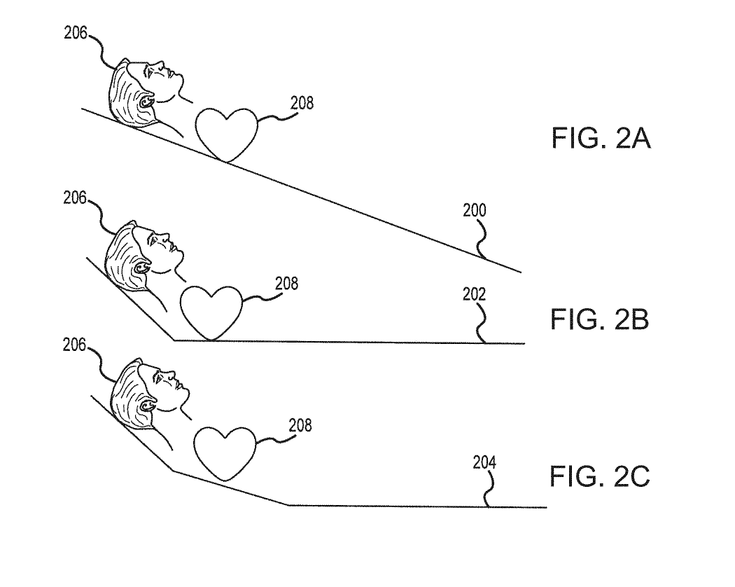

FIGS. 2A-2C demonstrate various set ups for HUP CPR as disclosed herein. Configuration 200 in FIG. 2A shows a user's entire body being elevated upward at a constant angle. As noted above, such a configuration may result in a reduction of coronary and cerebral perfusion during a prolonged resuscitation effort since blood will tend to pool in the abdomen and lower extremities over time due to gravity. This reduces the amount of effective circulating blood volume and as a result blood flow to the heart and brain decrease over the duration of the CPR effort. Thus, configuration 200 is not ideal for administration of CPR over longer periods, such as those approaching average resuscitation effort durations. Configuration 202 in FIG. 2B shows only the patient's head 206 being elevated, with the heart and thorax 208 being substantially horizontal during CPR. Without an elevated thorax 208, however, systolic blood pressures and coronary perfusion pressures are lower as lungs are more congested with blood when the thorax is supine or flat. This, in turn, increases pulmonary vascular resistance and decreases the flow of blood from the right side of the heart to the left side of the heart when compared to CPR in configuration 204. Configuration 204 in FIG. 2C shows both the head 206 and heart/thorax 208 of the patient elevated, with the head 206 being elevated to a greater height than that heart/thorax 208. In some embodiments, such as those where a method of CPR is performed that generates sufficient forward flow (such as ACD+ITD CPR) this elevation may be achieved by elevating the head 206 and heart/thorax 208 at a constant angle from the waist to the head. Such elevation results in lower right-atrial pressures while increasing cerebral perfusion pressure, cerebral output, and systolic blood pressure compared to CPR administered to an individual in the supine position, and may also preserve a central blood volume and lower pulmonary vascular resistance.

The type of CPR being performed on the elevated patient may vary. Examples of CPR techniques that may be used include manual chest compression, chest compressions using an assist device, either automated or manually, ACD CPR, a load-distributing band, standard CPR, stutter CPR, and the like. Such processes and techniques are described in U.S. Pat. Pub. No. 2011/0201979 and U.S. Pat. Nos. 5,454,779 and 5,645,522, all incorporated herein by reference. Further, various sensors may be used in combination with one or more controllers to sense physiological parameters as well as the manner in which CPR is being performed. The controller may be used to vary the manner of CPR performance, adjust the angle of inclination, the speed of head and thorax rise and descent, provide feedback to the rescuer, and the like. Further, a compression device could be simultaneously applied to the lower extremities or abdomen to squeeze venous blood back into the upper body, thereby augmenting blood flow back to the heart. Further, a compression-decompression band could be applied to the abdomen that compresses the abdomen only when the head and thorax are elevated either continuously or in a pulsatile manner, in synchrony or asynchronously to the compression and decompression of the chest. Further, a rigid or semi-rigid cushion could be simultaneously inserted under the thorax at the level of the heart to elevate the heart and provide greater back support during each compression.

Additionally, a number of other procedures may be performed while CPR is being performed on the patient in the torso-elevated state. For example, intrathoracic pressure regulation may be performed, which involves actively drawing air out of the lungs and/or preventing air from entering the lungs to control a patient's intrathoracic pressure, such as by using ACD+CPR, an impedance threshold device, a load distributing band, a ventilator, and the like. One such procedure is to periodically prevent or impede the flow in respiratory gases into the lungs. This may be done by using a threshold valve, sometimes also referred to as an impedance threshold device (ITD) that is configured to open once a certain negative intrathoracic pressure is reached. The invention may utilize any of the threshold valves or procedures using such valves that are described in U.S. Pat. Nos. 5,551,420; 5,692,498; 5,730,122; 6,029,667; 6,062,219; 6,810,257; 6,234,916; 6,224,562; 6,526,973; 6,604,523; 6,986,349; and 7,204,251, the complete disclosures of which are herein incorporated by reference.

Another such procedure is to manipulate the intrathoracic pressure in other ways, such as by using a ventilator or other device to actively withdraw gases from the lungs. Such techniques as well as equipment and devices for regulating respirator gases are described in U.S. Pat. Pub. No. 2010/0031961, incorporated herein by reference. Such techniques as well as equipment and devices are also described in U.S. patent application Ser. Nos. 11/034,996 and 10/796,875, and also U.S. Pat. Nos. 5,730,122; 6,029,667; 7,082,945; 7,310,649; 7,195,012; and 7,195,013, the complete disclosures of which are herein incorporated by reference.

In some embodiments, the angle and/or height of the head and/or heart may be dependent on a type of CPR performed and/or a type of intrathoracic pressure regulation performed. For example, when CPR is performed with a device or device combination capable of providing more circulation during CPR, the head may be elevated higher, for example 10-30 cm above the horizontal plane (10-45 degrees) such as with ACD+ITD CPR. When CPR is performed with less efficient means, such as manual conventional standard CPR, then the head may be elevated less, for example 5-20 cm or 5 to 20 degrees.

In some embodiments, an elevation device, such as those described in U.S. application Ser. No. 15/850,827 and U.S. application Ser. No. 15/601,494 (previously incorporated by reference), may be programmed to perform split-phase HUP CPR as described herein. For example, an elevation device may include a base and an upper support coupled with the base. The upper support may be configured to elevate an individual's heart, shoulders, and head relative to horizontal. In some embodiments, the upper support may include a single support surface that is configured to elevate the heart and head at a single angle, such as by bending the patient at or near the waist. In other embodiments, the upper support may include multiple support surfaces that may elevate the heart and head at different angles. The upper support may include an adjustment mechanism that is configured to adjust a degree of elevation of the upper support. For example, a motor or other actuator may be used to drive the angular and/or height adjustment of the upper support relative to the base. In some embodiments, a controller may be coupled with the adjustment mechanism and may be used to control the elevation of the upper support. For example, the controller may execute instructions that determine when and to what degree the adjustment mechanism adjusts the elevation position of the upper support. This may be based off of timing instructions that are derived from empirical studies and/or the elevation may be controlled based on one or more physiological parameters measured by one or more sensors that are in communication with the controller. For example, blood flow sensors, blood pressure sensors, end tidal CO₂ sensors, cerebral oximetry sensors, and/or sensors that monitor other physiological parameters that correlate directly or indirectly with cardio-pulmonary circulation and perfusion may be connected to the controller such that the controller may make adjustments in elevation degree and/or timing based on the sensed parameters.

The elevation device may also include a chest compression device, such as an automated chest compression device. In some embodiments, the chest compression device may be a load distributing band, a piston-based chest compression device, a combination of a load distributing band and an active decompression device, and/or other automated and/or manually actuated chest compression device. In some embodiments, the chest compression device may be configured to actively decompress the individual's chest between each compression such that the chest compression device may be used in the performance of ACD-CPR. In some embodiments, the controller (and/or another controller) may be coupled with the chest compression device such that the controller may control a rate and/or timing of the chest compressions being delivered to an individual according to the sequential elevation procedures described herein. The compression depth, decompression depth, rate of compression and decompression, and/or duty cycle may be varied based on a particular individual and/or based on measurements from the physiological sensors. In each of these examples, the head and thorax can also be lowered if clinically required, in some cases rapidly in less than 6 seconds.

A ventilation device that is coupled with one or more sensors may be included that is used to deliver positive pressure breaths to the individual at specific times during the CPR procedure. For example, the ventilation device may be configured to deliver positive pressure breaths only (or primarily) during the decompression phases of CPR, whether active or passive decompression is being utilized. The ventilation device may deliver the positive pressure breaths until the sensor(s) detect a compression phase of CPR. This may be done using any number of techniques. For example, the one or more sensors may detect a rise in intrathoracic pressure from chest compressions. In some embodiments, it may be determined that a compression phase is occurring when the intrathoracic pressure exceeds a threshold value, such as about 10-20 mmHg, with values below this threshold indicating a decompression phase. In some embodiments, a pressure or force sensor attached to the chest may be used to detect the compression and/or decompression cycle, such as by determining a direction, timing, duration, and/or magnitude of force exerted on the chest. For example, upward forces may indicate the decompression phase while downward forces may indicate the compression phase. Changes in the force over time, such as decreasing forces in either direction may indicate that the end of a particular phase is approaching, which will indicate a transition to the opposite phase of CPR. In some embodiments, the detection of compression and/or decompression phases may be done by sensing a change in electrical impedance across the chest and/or using a pressure or force sensor attached to some part of the patient's body or to the machine that is delivering the CPR or to the bed or platform that the patient is resting on. In some embodiments, chest compression and decompression cycles could be sensed by force, flow, and/or pressure transducers in a device attached to the airway such as an endotracheal tube or impedance threshold device, or an active intrathoracic pressure regulator device, the latter which simultaneously is used to regulate airflow in and out of the lungs and/or intrathoracic pressures. It will be appreciated that any combination of techniques may be used to sense the compression and/or decompression phases of CPR in accordance with the present invention.

In some embodiments, rather than (or in addition to) detecting the compression phase using one or more measured physiological parameters, the one or more sensors may include timing mechanisms that connect the chest compression device and the ventilation device to gate the control the timing of the ventilations relative to the compression/decompression cycle of the chest compression device.

Information from the sensed compression and decompression cycle is then used to regulate a timer either on a manual or automated positive pressure generating machine. In this manner, during each decompression phase, a positive pressure breath can be delivered over a duration of time sufficient for the delivery of the tidal volume. However, as soon as the compression phase is sensed (using one or more of the techniques described above) the positive pressure ventilation delivery is halted until the start of the next decompression phase, at which point the ventilation device completes the delivery of the breath. In other words, the chest compression device and ventilation device work in conjunction so that the delivery of the breath is split at least once, if not twice, and synchronized with the decompression phase of the chest compression device, whether active or passive. Thus, the delivery of the breath may initially increase intrathoracic pressure to about 10-20 cm of water, stop for less than one second during the compression phase, and then resume at the start of the next decompression phase. In this way a positive pressure breath can be delivered periodically (4-16 times per min) during CPR without limiting the tidal volume delivered, causing excessively high peak inspiratory pressures, or stopping chest compressions for the delivery of a breath.

The duration of the breath supplied by the ventilation device can be regulated depending on the physiological needs of the patient but in general the breath would be delivered over about 0.75 to 2.0 seconds with part of the breath delivered before each compression and the other part of the breath delivered after the compression phase. The duration of the breath and the tidal volume and the speed at which it is delivered could potentially vary from one breath to the next depending upon a feedback loop that is intended to maximize blood flow to one or more vital organs.

In some cases, rather than the entire positive pressure breath being delivered during the decompression phase, a majority of the positive pressure breath is delivered during the decompression phase with the remaining breath extending into a portion of the compression phase. For example, between about 70-90% (oftentimes around 80%) of each breath may be delivered during the decompressions phase while between about 10-30% (oftentimes around 20%) may be delivered during a subsequent compression phase. This may depend upon a sensed physiological measurement, for example, the intrathoracic pressure or airway pressure. Additionally, in some embodiments, a single positive pressure breath may be delivered across multiple decompression phases and/or compression phases.

In some embodiments, an impedance threshold device may be used to restrict the inflow of respiratory gases into the lungs except when a positive pressure breath is delivered. Sensors of the impedance threshold device and/or in communication with the impedance threshold device are used to determine the start of the chest compression and/or duty cycle and can also help to regulate the split phase ventilation either directly or by feedback to the ventilator.

In more sophisticated systems, the ventilation device and/or sensors in communication with the ventilation device may be configured to sense that the patient is trying to take a breath, a so-called agonal respiration or gasp. In some embodiments the delivery of the positive pressure breath would still be limited to only the period of time when the chest wall is recoil occurs either passively or actively during CPR. It is further noted that the ventilation device could be adjusted so that the timing and amount of the breath, in terms of the speed and waveform of volume delivery, could also be altered based upon the physiological needs of the patient. Moreover, this may vary when the patient is flat versus with head and thorax elevation and it may also very depending on the method of a CPR used as well as the method used to regulate intrathoracic pressure. In some cases it may be advantageous to provide split phase ventilation but instead of generating a negative intrathoracic pressure during the decompression phase with the addition of an intrathoracic pressure regulator device the intrathoracic pressure may be slightly positive (1-5 cm H2O) during portions or all of the decompression phase.

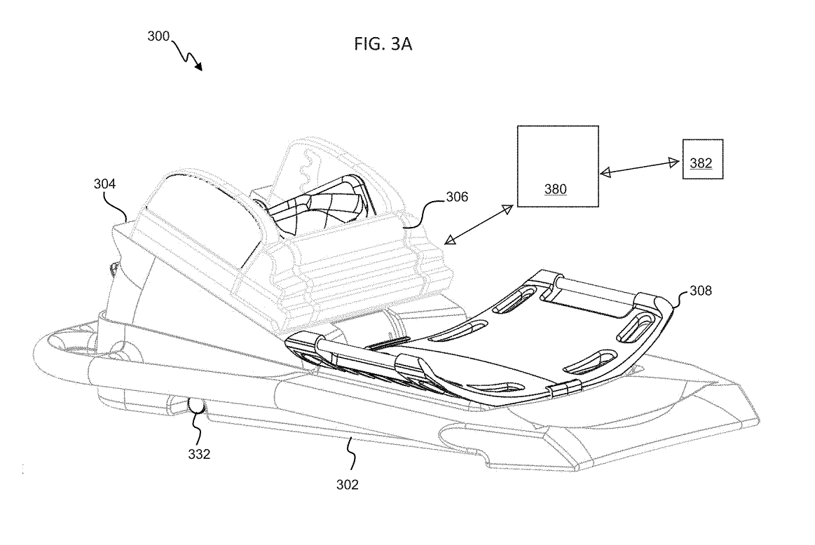

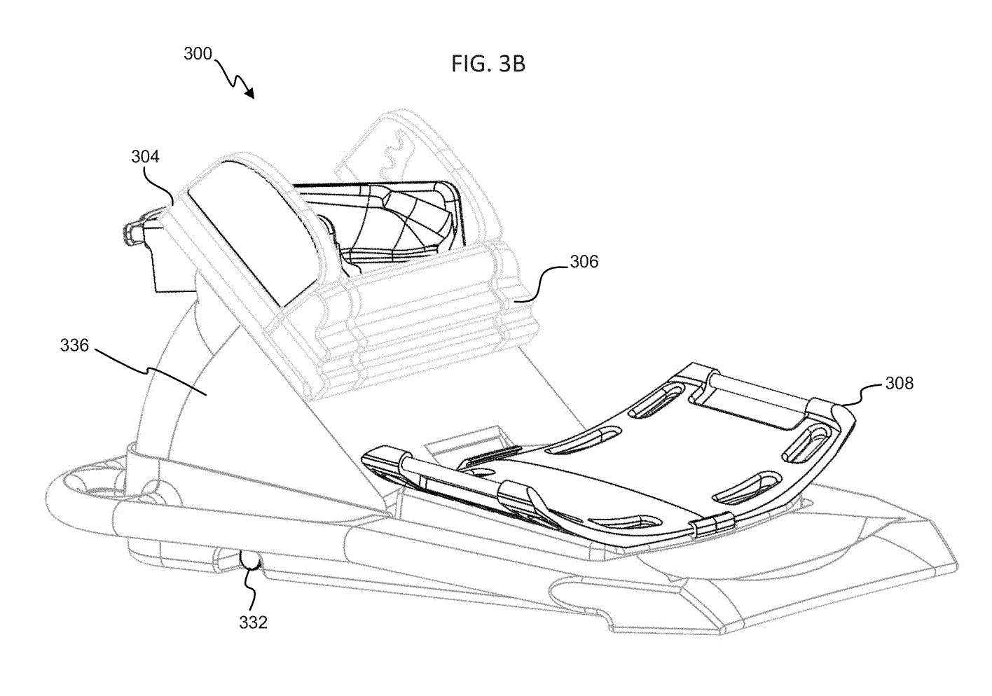

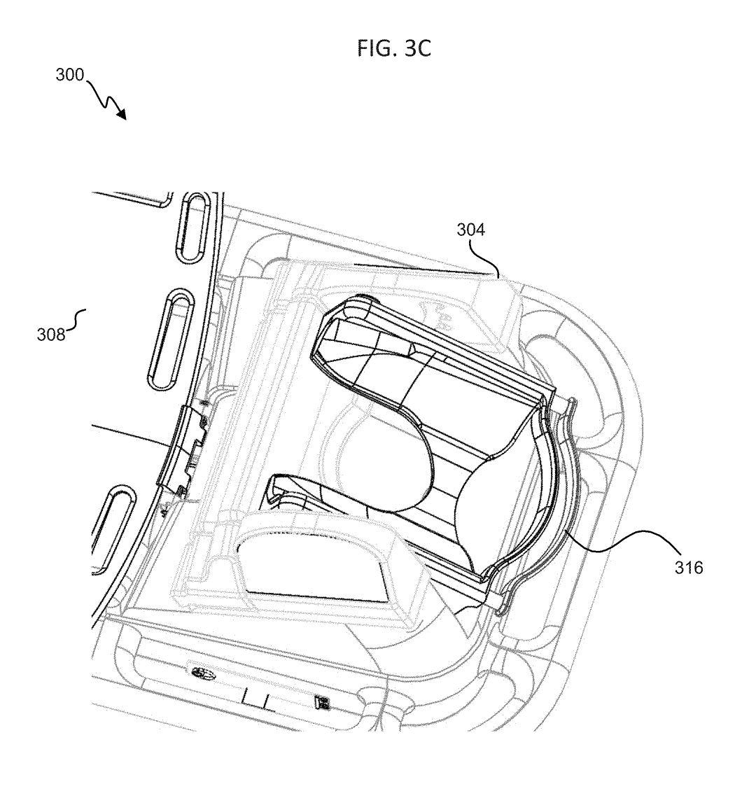

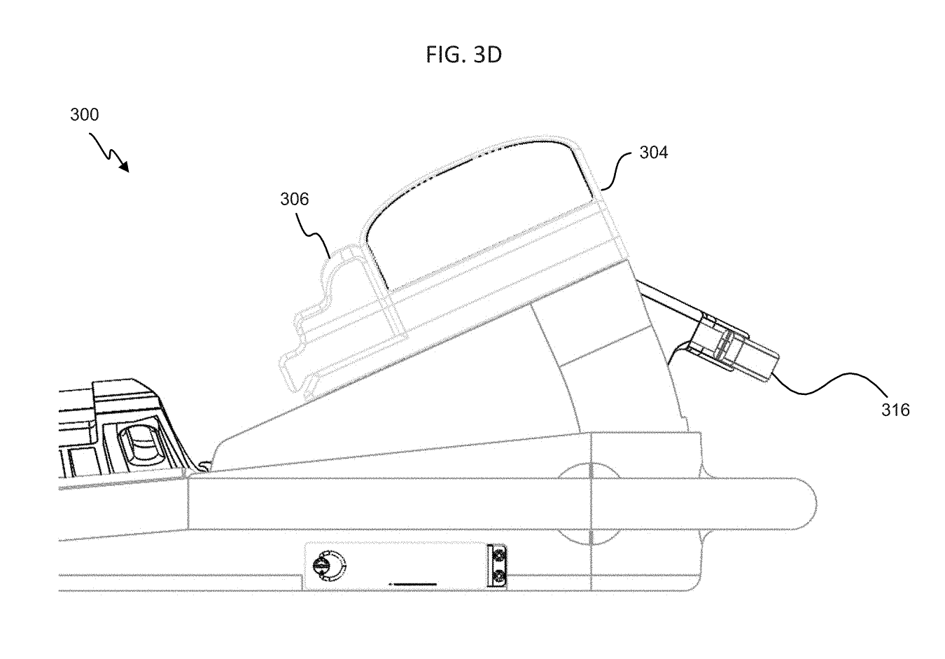

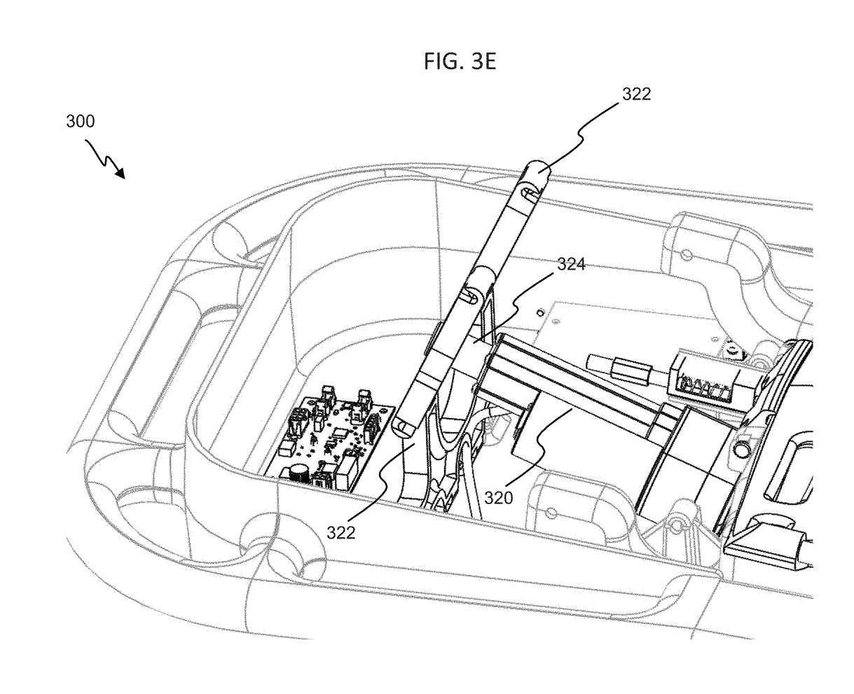

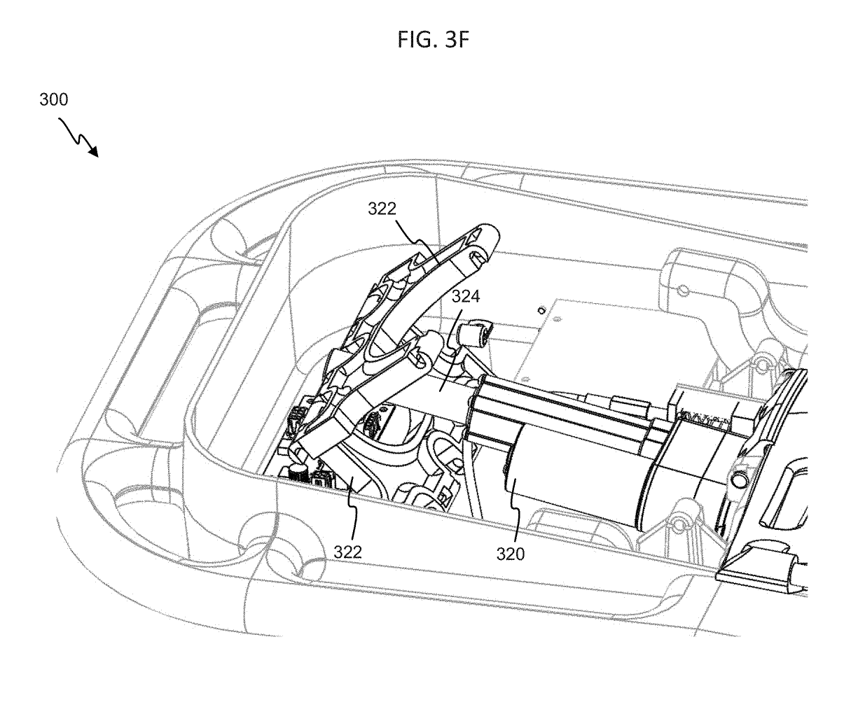

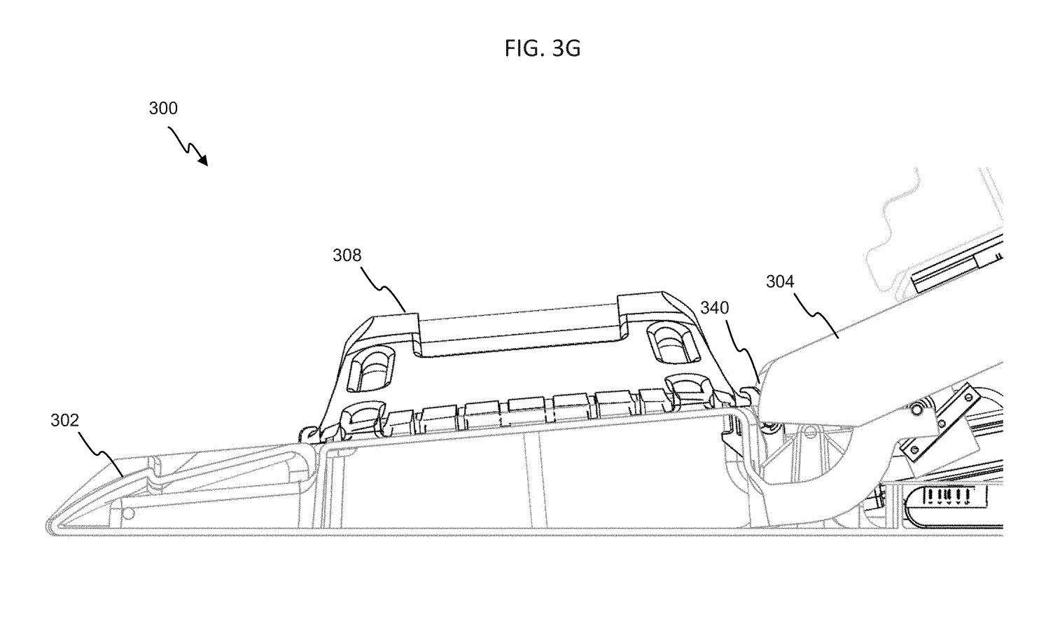

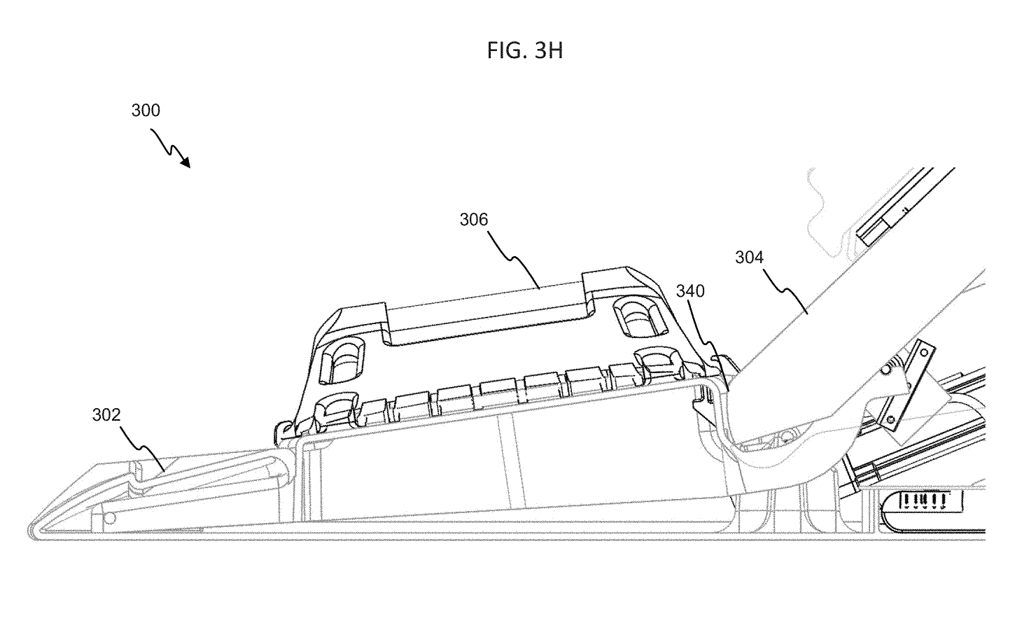

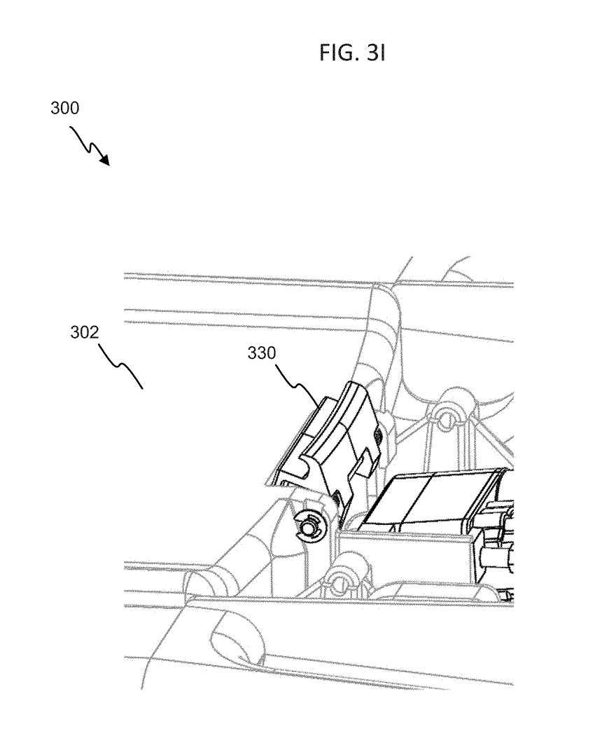

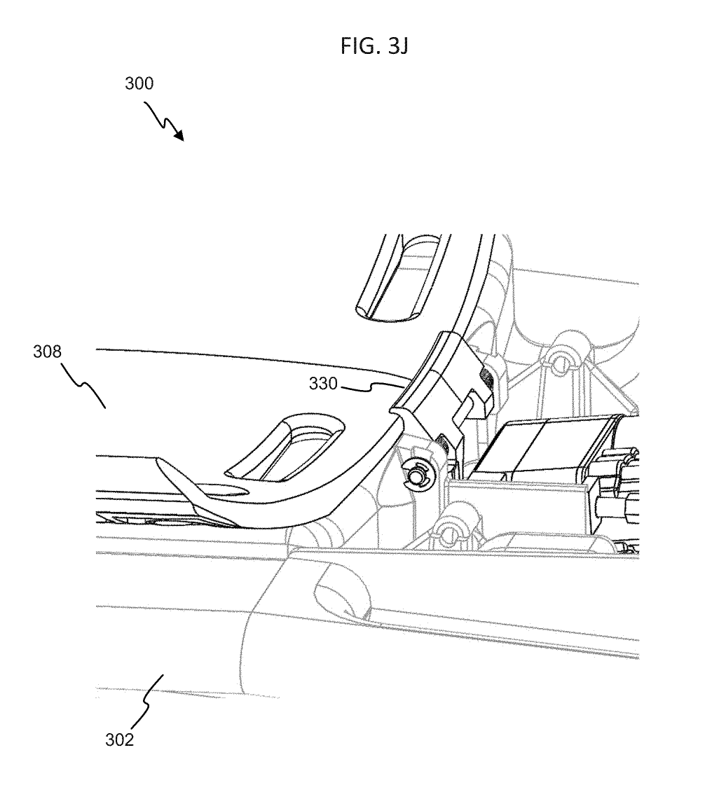

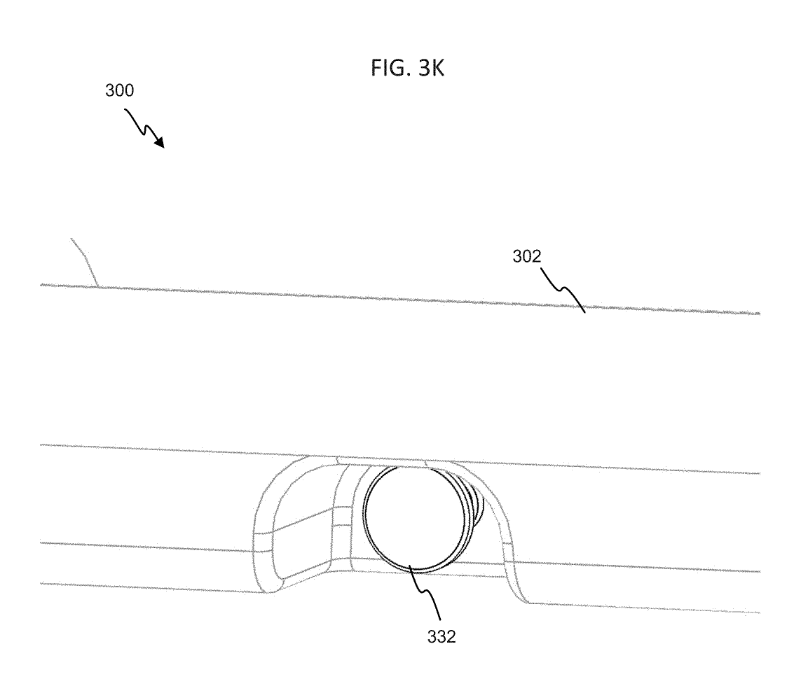

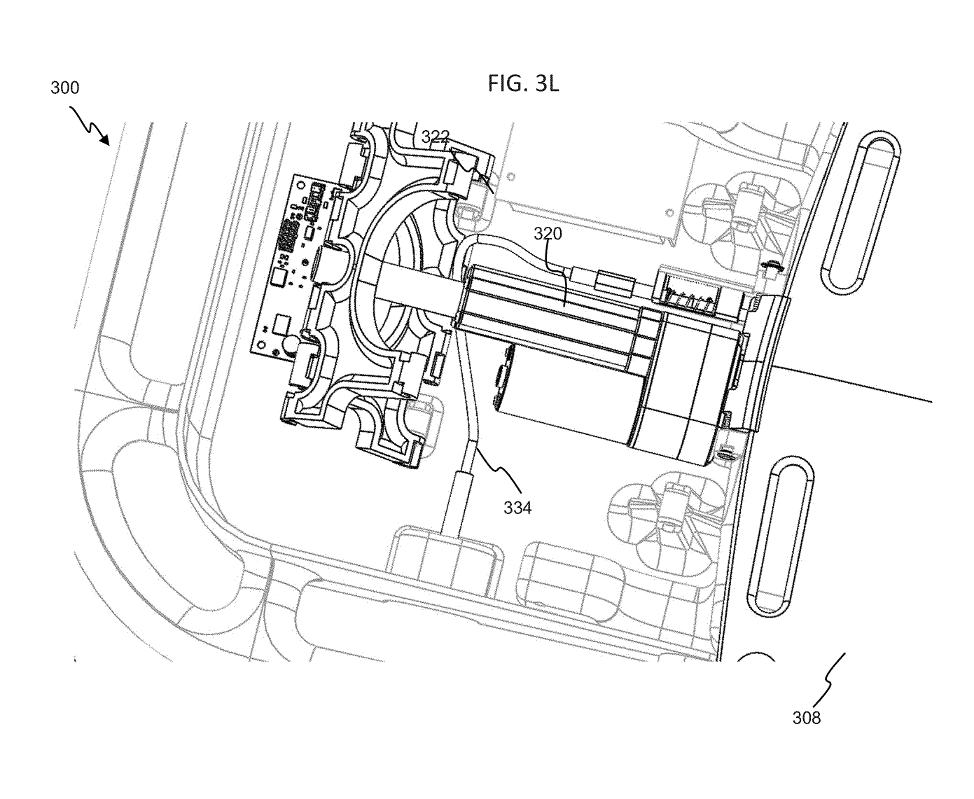

FIGS. 3A-3L depict an example of an elevation device 300, which may be similar to other elevation devices described herein. This device is designed to be placed under the patient, for example, as soon as a cardiac arrest is diagnosed. It has a low profile designed to slip under the patient's body rapidly and easily. For example, FIG. 3A shows that elevation device 300 may include a base 302 that supports and is pivotally or otherwise operably coupled with an upper support 304. Upper support 304 may include a neck pad or neck support 306, as well as areas configured to receive a patient's upper back, shoulders, neck, and/or head. An elevation mechanism may be configured to adjust the height and/or angle of the upper support 304 throughout the entire ranges of 0° and 45° relative to the horizontal plane and between about 5 cm and 45 cm above the horizontal plane. In some embodiments, the upper support 304 may be configured to elevate the middle of the patient's head to a height that is between about 2 and 42 cm above a middle of the heart. In some embodiments, an angle between the middle of the patient's head and a middle of the heart is between about 10 and 40 degrees relative to horizontal.

A user may be positioned on the elevation device 300 with his neck positioned on the neck support 306. In some embodiments, the neck support 306 may contact the individual's spine at a location near the C7 and C8 vertebrae. This position may help maintain the individual in the sniffing position, to help enable optimum ventilation of the individual. In some embodiments, the individual may be aligned on the elevation device 300 by positioning his nipples just above a center line of the back plate 308. The chest compression device is coupled with the back plate 308 such that the chest compression device is in alignment with the individual's sternum at a generally orthogonal angle to ensure that the chest compressions are delivered at a proper angle and with proper force. In some embodiments, the alignment of the chest compression device may be achieved may configuring the chest compression device to pivot and/or otherwise adjust angularly to align the chest compression device at an angle substantially orthogonal to the sternum.

As shown in FIG. 3A, elevation device 300 is in a lowered position, with the upper support 304 being configured to maintain the patient's head at a position that is slightly elevated relative to the heart, which is supported by back plate 308. In the lowered position, a head-receiving portion of the upper support 304 (which is designed to maintain the patient in the sniffing position and extends downward from a top surface of the upper support 304) maintains the base of the head at a generally horizontal level (within about 5 degrees) when in a fully lowered position. The upper support 304 may be raised as shown in FIG. 3B to elevate the patient's head, shoulders, and/or heart, which the head being supported at heights of between about 5 cm and 45 cm relative to a horizontal support surface on which the base 302 is supported. Upper support 304 may be configured to be adjustable such that the upper support 304 may slide along a longitudinal axis of base 302 to accommodate patients of different sizes as well as to accommodate movement of a patient associated with the elevation of the head by upper support 304. Without such sliding ability, a patient's upper body has a tendency to curl forward on the elevation device 300 as the patient's upper body is elevated. As shown in FIG. 3B, the upper support 304, including neck support 306, are extended away from the back plate 308 when the upper support 304 is elevated. In some embodiments, this sliding movement may be locked once an individual is positioned on the elevated upper support 304. In some embodiments, the upper support 304 may include one or more springs that may bias the upper support 304 toward the torso. This allows the upper support 304 to slide in a controlled manner when the individual's body shifts during the elevation process. In some embodiments, the one or more springs may have a total spring force of between about 10 lb. and about 50 lbs., more commonly between about 25 lb. and about 30 lb. Such force allows the upper support 304 to maintain a proper position, yet can provide some give as the head and upper torso are elevated. Further, the elevation device may include a slide mechanism such that with elevation of the head and neck the portion of elevation device behind the head and shoulder elongates. For example, the slide mechanism may include roller bearings that are mounted on a track that allows the upper support 304 to slide to accommodate patients of different sizes as well as to handle shifting of the body during elevation, which helps to maintain the neck in the sniffing position. In some embodiments, such as those shown in FIGS. 3C and 3D, a locking handle 316 is provided that allows medical personnel to adjust a lateral position of the upper support 304 relative to the base 302. To actuate the handle 324, a user must apply force to push a distal portion 326 of the handle 324 toward a fixed, proximal portion 328 of the handle 316. This action pushes a locking member (not shown) into a free space of a ratchet mechanism, allowing the user to adjust the lateral position of the upper support 304. Once released, the locking member may enter a tooth of the ratchet to set a position of the upper support 304 based on a size of the user. The upper support 304 may then only slide in small amounts to handle the shifting of the patient throughout the elevation process.

FIGS. 3E and 3F depict a linear actuator 320 that is used to raise and lower the upper support 304. Linear actuator 320 is coupled at a joint formed between two or more support members 322. Support members 322 are coupled between the base 302 and a bottom surface of the upper support 304 such that top support member(s) 322 is coupled with the upper support 304 and the bottom support member(s) 322 is coupled with the base 302. As linear actuator 320 is operated, a rod 324 of the linear actuator 320 shortens to draw the joint of the support members 322 toward the back plate 308, which causes an angled between the top and bottom supper members 322 to increase, such as shown in FIG. 3E, forcing the upper support 304 upward to elevate a patient's upper body. When operated in reverse, the rod 324 of linear actuator 320 extends, pushing on the joint to decrease the angle between the top and bottom support members 322 as shown in FIG. 3F, thereby lowering the upper support 304. It will be appreciated that the direction of operation of the linear actuator 320 and support members 322 may be reversed in some embodiments such that lengthening rod 324 causes elevation of the upper support 304 and shortening of rod 324 causes the lowering of the upper support 304. While shown here with a linear actuator 320 and support member 322 elevation mechanism, it will be appreciated that elevation device 300 may additionally or alternatively include other elevation mechanisms, such as threaded rods, lead screws, pneumatic and/or hydraulic actuators, motor driven telescopic rods, other elevation mechanisms, and/or combinations thereof.

Turning back to FIGS. 3A and 3B, the back plate 308 may be sized and shaped to receive a portion of the patient's back, just behind the heart and may be configured to couple with a chest compression device (not shown). Examples of CPR assist devices that could be used with the elevation device (either in the current state or a modified state) include the Lucas device, sold by Physio-Control, Inc. and described in U.S. Pat. No. 7,569,021, the entire contents of which is hereby incorporated by reference, the Defibtech Lifeline ARM-Hands-Free CPR Device, sold by Defibtech, the Thumper mechanical CPR device, sold by Michigan Instruments, automated CPR devices by Zoll, such as the AutoPulse, as also described in U.S. Pat. No. 7,056,296, the entire contents of which is hereby incorporated by reference, the Weil Mini Chest Compressor Device, such as described in U.S. Pat. No. 7,060,041 (Weil Institute), and the like. Chest compression devices used in accordance with the present invention may be configured to compress and/or actively decompress the chest.

In some embodiments, the back plate 308 may have a curved profile that may provide some flexibility to the back plate 308. This flexibility helps when the elevation device 300 is used in conjunction with a chest compression device, as the flexibility ensures that the right amount force applied to the patient's chest. For example, a central portion of the back plate 308 may flex in the presence of excessive force, thereby absorbing some of the force. For example, as a plunger of a chest compression device is pressed into the patient's chest, some force is transmitted through the patient to the back plate 308. The back plate 308 may be configured to bend away from the patient if this transferred force exceeds a threshold. This allows for the delivery of compression at the appropriate depth for patients with differing chest wall sizes and stiffness's. This helps prevent broken ribs and/or other injuries to the patient caused by too much force being applied to the patient's chest, as the flexing back plate 308, rather than the ribs or other body structures, absorbs a significant portion of the excess force. Such a design is particularly useful when the elevation device is used in conjunction with a chest compression device such as the Lucas device, sold by Physio-Control, Inc. and/or the Zoll AutoPulse.

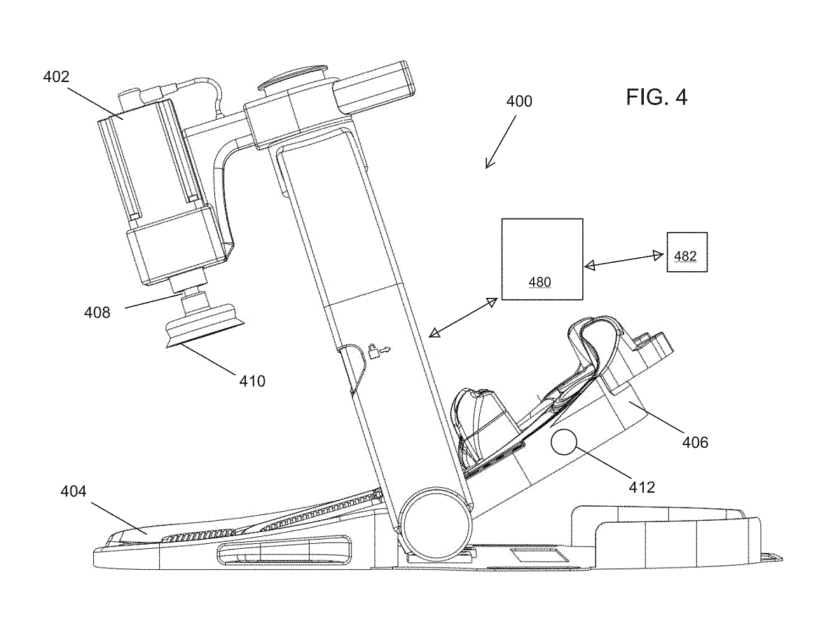

In some embodiments, the back plate 308 that is part of and/or is coupled with the upper support 304 in such a manner that an angle of the back plate 308 is adjustable relative to the base 302 and/or the upper support 304. The back plate 308 may be configured to adjust angularly to help combat thoracic shift to help maintain a chest compression device at a generally orthogonal to the sternum. The adjustment of the back plate 308 may create a separate elevation plane for the heart, with the head being elevated at a greater angle using the upper support 304 as shown in FIG. 3B. In some embodiments, the back plate 308 may be adjusted independently, while in other embodiments, adjustment of the back plate 308 is tied to the elevation of the upper support 304. For example, a back plate may include a roller (such as a v-groove bearing) positioned on an elevation track formed on or coupled with an underside of an upper support as illustrated in the embodiment discussed in relation to FIGS. 4G-4J of U.S. patent application Ser. No. 15/850,827, previously incorporated by reference. The roller may be positioned on a forward, raised portion of the elevation track. As the upper support 304 is elevated, the roller is forced upward by elevation track, thereby forcing an end of the back plate 308 proximate to the upper support 304 upwards. This causes the back plate 308 to tilt, thus maintaining the chest at a generally orthogonal angle relative to the chest compression device that is coupled with the back plate 308. Oftentimes, elevation track may be slanted from a raised portion proximate to the back plate 308 to a lowered portion. The elevation track may be tilted between about 4° and 20° to provide a measured amount of tilt relative to the thoracic shift expected based on a particular elevation level of the upper support 304. Typically, the back plate 308 will be tilted at a lower angle than the upper support 304 is inclined. Such simultaneous movement is also demonstrated in FIGS. 3G and 3H. In FIG. 3G, the upper support 304 is in the lowered position and the back plate 308 is in its original position. In FIG. 3H, the upper support 304 is elevated, which has caused the back plate 308 to have a corresponding forward tilt, which is less that than the degree of elevation of the upper support 304.

In some embodiments, the back plate may be removably coupled with the base 302 and/or the upper support 304. As shown in FIG. 3I, a latch 330 is provided beneath the back plate 308. The latch 330 may be spring biased such that a bottom surface of the latch 330 is able to receive a back edge of the back plate 308. The latch 330 may be pushed downward with the back plate 308 secured by a tip of the latch 330 until a spring-biased pin (not shown) slides along a bottom surface of the latch 330 and engages with a hole formed within a body of the latch 330. The pin secures the latch 330 in a locked position in which the back plate 308 is securely coupled with the base 302 and/or upper support as shown in FIG. 3J. A release knob 332 shown in FIG. 3K is coupled with the base 302 and may be used to draw the pin out of the hole formed in the body of latch 330 to release the back plate 308. For example, as shown in FIG. 3L, release knob 332 may be coupled to the pin via a flexible cable 334, similar to a brake cable on a bicycle. When knob 332 is pulled, the pin is drawn out of the hole and the spring force can push the latch 330 into a release position in which the back plate 308 may be removed from the base 302.

In some embodiments, the elevation device 300 may include a number of features that make the device more safe to operate. For example, as seen in FIG. 3A, elevation device 300 may include a vinyl (or other natural or synthetic material) cover 336 that may cover the moving components, such as the motor or actuator and/or slide mechanism. the cover 336 can extend and retract as the upper support 304 raises and lowers. For example, cover 336 may operate in a manner similar to a convertible top for an automobile, and may retract in a compact, accordion style manner when the upper support 304 is lowered. The upper support 304 of elevation device 304 may have a front surface 340 that is curved in a manner such that as the upper support 304 raises and lowers the front surface 340 stays approximately the same distance from the back plate 308. In other words, a gap between the two components remains generally constant, which eliminates any possible pinching hazard that could exist due to the relative movement between the two components.

In one embodiment, a controller and/or control system may adjust an actuation speed of a motor or other elevation mechanism to raise or lower the upper support 304 of the elevation device 300 within the necessary time frame. For example, medical personnel may set a desired elevation time, starting elevation angle, intermediate elevation angle(s), final elevation angle, rate of elevation, etc. The controller will then operate linear actuator 320, a motor, and/or other elevation mechanism to slowly raise the upper support 304 from a starting elevation angle to a final elevation angle over the selected time period, in one or more sequences. For example, the controller may be configured to elevate the head and thorax may be done in a sequence by 1) elevating the head and thorax over two or more sequential elevation steps and/or 2) elevating the head and thorax over a more prolonged period of time from the start of the elevation to the final height. In some embodiments, the controller may cause the chest compression device to perform CPR for a period of time (between about 30 seconds and 10 minutes, more commonly between about 2 minutes and 8 minutes, and more commonly between about 3 minutes and 6 minutes) while the individual is in a flat, supine position (or nearly supine, such as with the head and/or heart elevated slightly to an angle of less than about 5 degrees relative to horizontal) prior to causing the actuator to elevate the upper support 304 and the individual to an intermediate and/or final height. In some embodiments where the individual has been primed flat, the controller may perform an additional priming step at an intermediate elevation position prior to elevating the individual to the final/highest elevation position. In other embodiments, the individual may be primed by first elevating the individual's head and heart to one or more intermediate elevation positions (i.e. between about 10 and 25 degrees) and then performing chest compressions for a period of time prior to elevating the individual's heart and head to a final elevation position (i.e. between 20 and 45 degrees). The chest compressions may be continued during the elevation adjustment periods after each priming step.

The controller may also control the rate of elevation of the upper support 304. As just one example, the controller may maintain the elevation speed at a rate of not faster than 1⁰ over each 3 second period. The lift speed may be linear and/or non-linear throughout each elevation step.

Blood drains rapidly from the head when the patient has no blood pressure and the head and upper body are elevated. As a result, there is a need to lower the head fairly rapidly to prevent blood loss in the brain if CPR is stopped while the head is elevated. Typically, this means that the patient's head and upper body may be elevated at a different rate than it is lowered. The patient's head may be lowered by the controller between about 1 and 10 seconds, and typically between about 2-8 seconds.

The controller may also be configured to cause the actuator to slowly and continuously raise the upper support 304 (and individual's heart, shoulders, and head) from a starting elevation position to a final elevation position. For example, a starting elevation position may include the individual being positioned in a generally flat, supine position (with the head elevated less than 5° relative to horizontal). The individual's head, shoulders, and heart may be slowly raised (linearly and/or non-linearly) from the starting elevation position to a position where the head is elevated between about 20 and 45 degrees relative to horizontal (an absolute elevation of the heart by about 5-10 cm and an absolute elevation of the head by about 15-25 cm, although these ranges may vary based on the age, size, and/or physiology of a specific individual) over a period of between about 30 seconds and 10 minutes, more commonly between 1 minutes and 4 minutes, and optimally between about 1 minutes and 1 minutes, while CPR is performed. For example, the head, shoulders, and heart may be raised at a rate of between about 2.25°/second and about 1.5°/minute. In other embodiments, an individual may be quickly raised to a starting elevation position of between about 8-15 degrees before slowly elevating the head, shoulders, and heart to a final elevation positon over a period of between about 30 seconds and 10 minutes, more commonly between 2 minutes and 8 minutes, and optimally between about 3 minutes and 6 minutes, while CPR is performed.

In some embodiments, the controller may receive data from one or more physiological sensors and use this data to determine rates and timing of elevation and lowering. For example, the patient on the elevation device 300 may be monitored using an electrocardiogram (ECG). The ECG may detect a stable heart rhythm even if the individual has no palpable pulse. Based on this detection of the stable heart rhythm, it may be determined to stop the performance of chest compressions and to promptly lower the upper support 304. For example, once it is detected that the patient has a stable heart rhythm, the controller may alert medical personnel that chest compressions should be ceased, and may send a signal to the motor or other actuator to cause the upper support 304 to rapidly lower. In some embodiments, alerting medical personnel may involve producing a visual indicator, such as lighting up a light emitting diode (LED) or other light source and/or presenting a textual and/or image-based display on a screen of the elevation device 300. In one embodiment, upon detecting a stable heart rhythm, the controller may send a command to the automatic chest compression device that causes the chest compression device to stop the delivery of chest compressions and/or decompressions. In another embodiment, upon detecting the stable heart rhythm, the controller will alert medical personnel, who may then operate the elevation device 300 to lower the upper support 304. It will be appreciated that other sensors may be used in conjunction with the elevation device 300 to determine: when to start and/or stop CPR, when to elevate and/or lower a patient's upper body, a degree of elevation of the patient's upper body, a rate of elevation or lowering of the patient's upper body, and/or other parameters of CPR and/or ITPR.

The elevation device 300 elevates the head above the heart, with the level of elevation optionally varying depending upon the method of CPR. Conventional closed chest manual CPR itself is inherently inefficient, providing only about 20% of normal blood flow to the heart and brain. Elevation of the head is not safe during conventional CPR as it is not possible to consistently or safely push enough blood “uphill” to the head to take advantage of the effects of gravity of the venous side of the arterial-venous circuit that is integral to cerebral perfusion. Methods of CPR that generate the most forward flow provide the opportunity to elevate the head above the heart more than those methods that provide less forward flow. For example, active compression decompression (ACD) CPR with an impedance threshold device (ITD) can triple blood flow to the heart and brain compared with conventional manual CPR alone and therefore the head can be elevated higher and still get enough perfusion to take advantage of the effects of gravity with HUP CPR. By contrast, the head should not be elevated as much with conventional CPR and the ITD as forward blood flow without ACD CPR is less, and therefore too much elevation of the head could worsen outcomes. For these reasons the optimal head elevation may vary both depending upon the method of CPR used and the condition of the patient.

The relative vertical distance between the head and the heart is important as the amount of pressure needed to “lift” or pump the blood from the heart to the brain is related to this distance. Further, the vertical distance between the head and the heart affects the amount of cerebral perfusion. Although the amount of elevation of the head relative to the heart may vary depending upon the method of CPR (which is the mechanism used to pump the blood), it is generally preferred to have the head elevated relative to the heart by a distance in the range from about 2 cm to about 42 cm. In the specific case where ACD-CPR is being performed with an ITD, the distance may be in the range from about 5 cm to about 25 cm, for standard CPR with an ITD between about 5 cm and about 20 cm, for ACD CPR by itself between about 5 cm and about 20 cm, and with conventional or standard CPR between about 3 cm and about 15 cm. Further, the distance that the heart may be elevated relative to a support surface upon which the lower portion of the patient is resting (such as a table, floor, gurney, stretcher, or the ground) may be in the range from about 3 cm to about 20 cm (with ranges between about 4 cm and 10 cm being common), while the height of the head relative to the support surface may be in the range from about 5 cm to about 45 cm (with ranges between about 10 cm and 40 cm being common). When performing ACD-CPR+ITD, the distance that the heart may be elevated relative to a support surface upon which the patient is resting may be in the range from about 3 cm to about 20 cm, while the height of the head relative to the support surface may be in the range from about 5 cm to about 45 cm. Of course, these relative heights can also be thought of in terms of an angle of elevation of the upper body relative to the lower body when the patient is bent at the waist when performing CPR. Such angles are described herein. Typically, the angle between the patient's heart and brain is between 10 degrees and 40 degrees relative to horizontal to achieve the necessary elevation, although it will be appreciated that such angles are largely driven by the patient's physiology (height, distance between head and heart, etc.).

A ventilation device 380 may be included with the elevation device 300. Ventilation device 380 may be built into the elevation device 300, coupled with the elevation device 300, and/or may be a standalone device that may be used in conjunction with the elevation device 300 when performing split-phase CPR in a head up position. The ventilation device 380 may be any automated and/or manual device that may be interfaced with a patient's airway and that can deliver a positive pressure breath to the airways in a controlled manner. The ventilation device 380 is coupled with one or more sensors 382 that are used by the ventilation device to detect the compression and/or decompression phases of CPR such that the ventilation device 380 delivers positive pressure breaths to the individual at specific times during the CPR procedure. For example, the ventilation device 380 may be configured to deliver positive pressure breaths only (or primarily) during the decompression phases of CPR, whether active or passive decompression is being utilized. The ventilation device 380 may deliver the positive pressure breaths until the sensor(s) detect a compression phase of CPR. This may be done using any number of sensors 382, such as intrathoracic pressure sensors, pressure and/or force sensors attached to the chest electrical impedance sensors, pressure and/or force sensors attached to some part of the patient's body or to the chest compression device, force, flow, and/or pressure transducers in a device attached to the airway such as an endotracheal tube or impedance threshold device, or an active intrathoracic pressure regulator device, and/or timing sensors.

The ventilation device 380 may use the sensor data to control the timing of delivery of positive pressure ventilations. For example, during each decompression phase, a positive pressure breath can be delivered until the compression phase is sensed. The positive pressure ventilation delivery is then halted until the start of the next decompression phase, at which point the ventilation device completes the delivery of the breath and/or starts delivery of a new breath. The duration of the breath supplied by the ventilation device 380 during the decompression phase can be regulated depending on the physiological needs of the patient but in general the breath would be delivered over about 0.75 to 2.0 seconds with all or part of the breath delivered before each compression and any remaining part of the breath delivered after the compression phase.

In some cases, rather than the entire positive pressure breath being delivered during the decompressions phase, a majority of the positive pressure breath is delivered during the decompression phase with the remaining breath extending into a portion of the compression phase. For example, between about 70-90% (oftentimes about 80%) of each breath may be delivered by the ventilation device 380 during the decompressions phase while between about 10-30% (oftentimes about 20%) may be delivered during a subsequent compression phase. This may depend upon a sensed physiological measurement, for example, the intrathoracic pressure or airway pressure. Additionally, in some embodiments, a single positive pressure breath may be delivered across multiple decompression phases and/or compression phases.

In some embodiments the heart will not be elevated. For example, a small head-only elevation device may be used that would only elevate the head, while allowing the heart to remain in the horizontal plane along with the lower body. Such elevation devices may be particular useful when performing CPR without the use of a CPR assist device/automated chest compression device as it reduces the amount of force needed to pump blood to the patient's brain during CPR. In such cases, the head would be raised to a distance in the range from about 5 to 20 cm relative to the heart (which is not elevated relative to the support surface).

In some embodiments, the controller be configured to detect a type of CPR being delivered and may automatically adjust an elevation of the heart and/or head based on the detected level of force. This may be done, for example, by allowing a user to input a type of CPR being performed into the elevation device 300. In other embodiments, such as those where a chest compression device is coupled with or formed integrally with the elevation device, the elevation device may communicate with the chest compression device to determine if the chest compression device is being used to deliver compressions and/or an amount of force being delivered and may make any necessary elevation adjustments based on this data. In other embodiments, one or more physiological sensors may be used to detect physiological parameters, such as cerebral perfusion pressure, intrathoracic pressure, and the like. This sensor data may be used to determine a compression force and/or otherwise determine how high to elevate the head and heart.

In some embodiments, the controller system may be used to control the timing of the delivery of positive pressure breaths by the ventilation device 380. For example, the controller system may receive data from the chest compression device, one or more physiological sensors, and/or timing sensors to ensure that the ventilation device 380 and chest compression device operate synchronously with one another to deliver positive pressure breaths solely or primarily during the decompression phase of CPR in accordance with the present invention. In other embodiments, the ventilation device 380 may have its own dedicated controller system that may take in external data from one or more sources and/or sensors 382 (the chest compression device, one or more physiological sensors, and/or timing sensors) to control the timing of the delivery of positive pressure breaths to the patient.

A chest compression device may be permanently (which may include being formed integral with the elevation device) or removably coupled with an elevation device similar to those described elsewhere herein. For example, as illustrated in FIG. 4, chest compression device 402 is coupled with an elevation device 400. Elevation device 400 may be similar to elevation device 300 described above and may include any of the features described in relation to elevation device 300. For example, elevation device 400 may be configured to elevate an individual's heart, shoulders, and/or head in a controlled manner during HUP CPR. As just one example, elevation device 400 may include a base 404, an upper support 406 coupled with the base 404, and an adjustment mechanism (not shown), such as a motor, jack, and/or other manual and/or automated drive mechanism, that is coupled with the upper support 406 and may be actuated to adjust a degree and/or height of elevation of the upper support 406 relative to the base 404. The upper support 406 is configured to support an individual's heart, shoulders, and head relative to horizontal such that as the upper support 406 is elevated, it lifts the individual's heart, shoulders, and head.

In some embodiments, chest compression device 402 may be configured to vary the amount of active decompression applied to the patient's chest based on the elevation angle of the heart and/or head of the individual. This may include decompressing further, faster, or for a longer period of time over the course of the compression decompression cycle. Chest compression device 402 may be configured to vary the amount of active compression applied to the patient's chest based on the elevation angle of the heart and/or head of the individual. This may include compressing further, faster, or for a longer period of time over the course of the compression decompression cycle.

In some embodiments, the adjustment mechanism may be configured to slowly elevate the upper support 406 over a period of time and/or quickly lower the upper support 406 (such as over a period of 0.5 to 10 seconds). For example, individual's head, shoulders, and heart may be slowly raised (linearly and/or non-linearly) from a starting elevation position (which may be substantially flat and/or at an elevation angle of about 5 and 15 degrees relative to horizontal) to a position where the head is elevated between about 20 and 45 degrees relative to horizontal (an absolute elevation of the heart by about 5-10 cm and an absolute elevation of the head by about 15-25 cm, although these ranges may vary based on the age, size, and/or physiology of a specific individual) over a period of between about 30 seconds and 8 minutes, more commonly between 1 minute and 4 minutes, and optimally between about 1 minute and 3 minutes, while CPR is performed. For example, the head, shoulders, and heart may be raised at a rate of between about 0.1°/second and about 0.5°/second. In other embodiments, one or more intermediate elevations may be achieved. For example, the starting position may be with the individual and upper support 406 in a substantially horizontal position and the adjustment mechanism may raise the upper support 406 to an intermediate position (such as an elevation angle of the head of about 5 and 15 degrees) at a first elevation rate. After a period of time, the adjustment mechanism may then further elevate the upper support 406 to a second elevation position (where the head is elevated between about 20 and 45 degrees) at a second rate, which may be the same or different from the first rate. For example, the first rate may be between about 0.5°/second and 2.25°/second while the second rate may be between about 4.0°/minute and 40°/minute, although other ranges may be contemplated. For example, in some embodiments, the first rate may be slower than the second rate, while in other embodiments, the first rate may be quicker than the second rate. In some embodiments, rather than being elevated at a rate of a number of degrees per unit of time, the upper support 406 may be elevated at a rate of a distance per unit of time. As just one example, the first rate may be between about 0.25 cm and 2 cm/min and the second rate may be between about 3 cm and 30 cm/min. It will be appreciated that any number of elevation positions may be used in a HUP CPR procedure, with a noticeable pause in elevation being performed at each elevation position. it will be further appreciated that elevation device 400 may be operated in accordance with any of the elevation sequence disclosed in relation to elevation device 300.

A ventilation device 480 may be included with the elevation device 400. Ventilation device 480 may be built into the elevation device 400, coupled with the elevation device 400, and/or may be a standalone device that may be used in conjunction with the elevation device 400 when performing split-phase CPR in a head up position. The ventilation device 480 may be any automated and/or manual device that may be interfaced with a patient's airway and that can deliver a positive pressure breath to the airways in a controlled manner. The ventilation device 480 is coupled with one or more sensors 482 that are used by the ventilation device to detect the compression and/or decompression phases of CPR such that the ventilation device 380 delivers positive pressure breaths to the individual at specific times during the CPR procedure. For example, the ventilation device 480 may be configured to deliver positive pressure breaths only (or primarily) during the decompression phases of CPR, whether active or passive decompression is being utilized. The ventilation device 480 may deliver the positive pressure breaths until the sensor(s) detect a compression phase of CPR. This may be done using any number of sensors 482, such as intrathoracic pressure sensors, pressure and/or force sensors attached to the chest electrical impedance sensors, pressure and/or force sensors attached to some part of the patient's body or to the chest compression device, force, flow, and/or pressure transducers in a device attached to the airway such as an endotracheal tube or impedance threshold device, or an active intrathoracic pressure regulator device, and/or timing sensors.

The ventilation device 480 may use the sensor data to control the timing of delivery of positive pressure ventilations. For example, during each decompression phase, a positive pressure breath can be delivered until the compression phase is sensed. The positive pressure ventilation delivery is then halted until the start of the next decompression phase, at which point the ventilation device completes the delivery of the breath and/or starts delivery of a new breath. The duration of the breath supplied by the ventilation device 480 during the decompression phase can be regulated depending on the physiological needs of the patient but in general the breath would be delivered over about 0.75 to 2.0 seconds with all or part of the breath delivered before each compression and any remaining part of the breath delivered after the compression phase.

In some cases, rather than the entire positive pressure breath being delivered during the decompressions phase, a majority of the positive pressure breath is delivered during the decompression phase with the remaining breath extending into a portion of the compression phase. For example, between about 70-90% (oftentimes about 80%) of each breath may be delivered by the ventilation device 480 during the decompressions phase while between about 10-30% (oftentimes about 20%) may be delivered during a subsequent compression phase. This may depend upon a sensed physiological measurement, for example, the intrathoracic pressure or airway pressure. Additionally, in some embodiments, a single positive pressure breath may be delivered across multiple decompression phases and/or compression phases.

In some embodiments, elevation device 400 may include an elevation indicator 412 that allows users to monitor an elevation angle and/or height of the upper support 406, the individual's head, and/or the individual's heart. For example, the elevation device 400 may include a display screen, such as an LED and/or LCD screen that produces a digital indication of the current elevation angle and/or height. In other embodiments, indicia that allow a user to discern the elevation angle and/or height may be provided on a portion of the elevation device 400. For example, indicia may be painted, imprinted, embossed, affixed (such as a decal), and/or otherwise provided on a side of the elevation device 400 that allow a user to quickly determine the elevation position of the upper support 406. As just one example, the indicia may include angular positions, such as predetermined angular settings and/or marks that indicate a number of angular positions spaced at regular and/or irregular intervals. Alternatively, or in addition to the angular marks, the elevation device 400 may include markings that indicate a height of the upper support 406, such as markings at each cm, inch, and/or portion thereof.

As illustrated, chest compression device 402 may be configured to perform ACD-CPR and may include a piston-driven plunger 408 attached to a suction cup 410. Chest compression device 402 may be configured to apply successive compressions and decompressions in a uniform and symmetric manner. To achieve this, chest compression device 402 may be configured to maintain the plunger 408 at a substantially perpendicular angle relative to the patient's sternum, which may involve the plunger 408 being coupled with the upper support 406 such that as the upper support 406 elevates the patient's heart, the chest compression device 402 is elevated at a same angle. Suction cup 410 may be configured to be affixed to the patient's chest such that the chest compression device 402 may actively decompress the patient's chest, oftentimes to a level exceeding a neutral decompression position of the chest, after each compression of the chest. For example, after each chest compression, the plunger 408 and suction cup 410 may be drawn upward by a motor of the chest compression device. as the suction cup 410 is drawn upward, the suction cup applies a decompressive force to the chest that pulls the chest upwards, thereby creating a negative pressure within the chest between each decompression that draws blood back into the heart after each chest compression. It will be appreciated that an adhesive pad and/or other mechanism for grasping the patient's chest with enough force to lift the chest beyond a neutral decompression position may be used in place of (or in addition to) suction cup 410 in some embodiments. It will be appreciated that the duty cycle for the compression phase and decompression may be 50% or may vary. Similarly, it will be appreciated that the compression depth may be 5 cm as currently recommended by the American Heart Association or it may vary. The compression and decompression excursion distance or force may also vary depending upon the level of head and thorax elevation, the angle or head and thorax elevation, and/or as a result of a change in a physiological sensor that helps to regulate the elevation of the head and thorax and/or the compression/decompression force and excursion distance.

In some embodiments, chest compression device 402 may be configured to increase the amount of active decompression of the individual's chest as the heart and/or head are further elevated so as to increase the negative pressure within the chest as the patient's heart and/or head are elevated. The change in active decompression of the chest compression device 402 may be done in a linear and/or non-linear manner in relation to the elevation height and/or angle of the patient. The change in active decompression may be accomplished by increasing the amount of decompressive force applied to the chest by the chest compression device and/or by increasing a maximum decompression distance of the chest as the head and/or heart are elevated. For example, the motor of the chest compression device may be configured to apply more upward pulling force to the plunger 408 and suction cup 410 between each chest compression and/or may change the position of a mechanical (or electro-mechanical) stop of the upward translation of the plunger 408 to adjust the amount of decompressive force and/or distance applied to the chest.

Similarly, in some embodiments, chest compression device 402 may be configured to increase the amount of active compression of the individual's chest as the heart and/or head are further elevated so as to increase the positive pressure within the chest as the patient's heart and/or head are elevated. The change in active compression of the chest compression device 402 may be done in a linear and/or non-linear manner in relation to the elevation height and/or angle of the patient. The change in active compression may be accomplished by increasing the amount of compressive force applied to the chest by the chest compression device and/or by increasing a maximum compression distance of the chest as the head and/or heart are elevated. For example, the motor of the chest compression device may be configured to apply more downward force to the plunger 408 and suction cup 410 between each chest decompression and/or may change the position of a mechanical (or electro-mechanical) stop of the downward translation of the plunger 408 to adjust the amount of compressive force and/or distance applied to the chest.

The chest compression device 402 may include and/or be in communication (via wired and/or wireless connections) with one or more sensors, such as physiological sensors and/or elevation sensors, that may provide feedback that is used to drive the active decompression levels of the chest compression device 402. For example, the active decompression levels of the chest compression device may be based on an angle and/or height of elevation of the head and/or heart based on data measured by the elevation sensor(s). Additionally, or alternatively, the active decompression levels of the chest compression device may be based on a measured negative pressure and/or various physical parameters as measured by the physiological sensor(s). By using measured physiological parameters of an individual to drive the amount of active decompression of the chest compression device it may be possible to tailor the performance of ACD CPR to the needs of a particular individual, which may maximize the efficacy of the resuscitation procedure.

The elevation device 400 may include a controller system that includes one or more controllers that are configured to control the adjustment mechanism and/or chest compression device 402. For example, the controller system may be configured to prime an individual's circulatory system by causing the chest compression device 402 to perform chest compressions on the individual while the individual's heart, shoulders, and head are supported by the upper support 406 at a first elevation position for a period of time. The controller system may also be configured to cause the adjustment mechanism to adjust the degree of elevation of the upper support 406 to a second elevation position which is greater than the first elevation position after the period of time has elapsed while performing chest compressions. The controller system may be configured to cause the chest compression device 406 to perform chest compressions on the individual while the individual's heart, shoulders, and head are at the second elevation position.

In some embodiments, the controller system may also be configured to control the timing of the delivery of positive pressure breaths by the ventilation device 480. For example, the controller system may receive data from the chest compression device, one or more physiological sensors, and/or timing sensors to ensure that the ventilation device 480 and chest compression device operate synchronously with one another to deliver positive pressure breaths solely or primary during the decompression phase of CPR in accordance with the present invention. In other embodiments, the ventilation device 480 may have its own dedicated controller system that may take in external data from one or more sources (the chest compression device, one or more physiological sensors, and/or timing sensors) to control the timing of the delivery of positive pressure breaths to the patient.

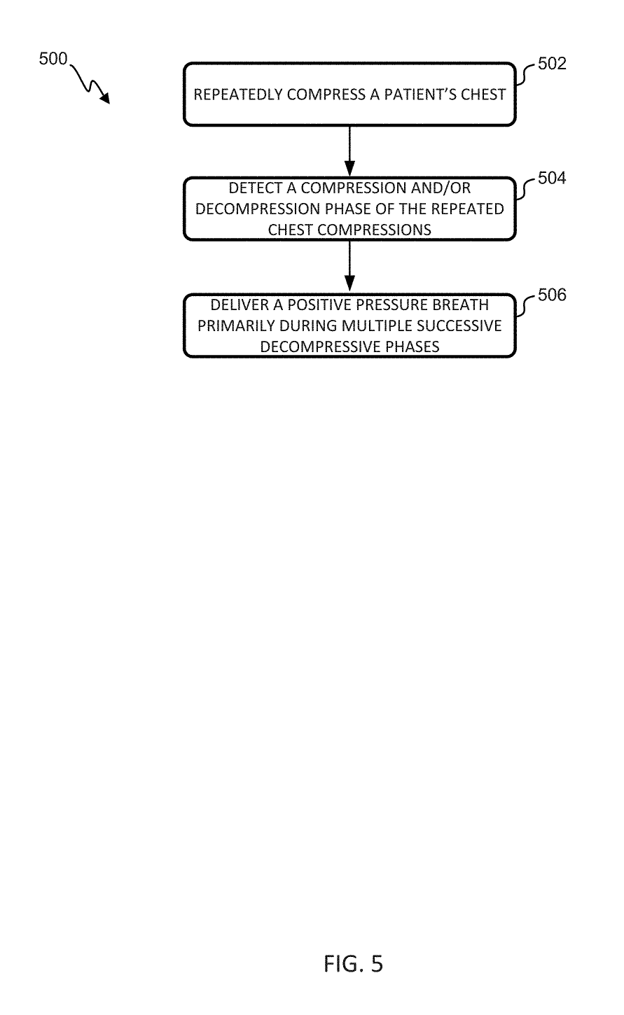

FIG. 5 is a flowchart depicting a process 500 for ventilating a patient during CPR. Process 500 may be performed using any of the ventilation devices, elevation devices, and/or chest compression devices described herein. Process 500 may begin at block 502 by repeatedly compressing the patient's chest resulting in repeating compression and decompression phases of the patient's chest. The chest compressions may be performed by a manual and/or automated chest compression device, such as chest compression device 402 described herein and/or may be performed manually. The chest compressions may be performed with the patient in a flat, supine position and/or with the patient's heart, shoulders, and head elevated relative to the lower body and the horizontal plane. At block 504, one or both of a compression phase and the decompression phase of the repeated compressions of the patient's chest may be detected. For example, feedback from one or more sensors attached to the patient, a chest compression device, a ventilation device, timing sensor, and/or other sensor may be used to determine when a compression phase and/or decompression phase of CPR is occurring. At block 506, a positive pressure ventilation may be delivered to the patient only or primarily during multiple successive decompression phases of the repeated compressions of the patient's chest. The delivery (and stopping) of the positive pressure ventilation is triggered based on the sensed compression and/or decompression phase. In some embodiments, the process 500 includes ceasing the delivery of the positive pressure ventilation upon the detection of the compression phase. Process 500 may further include detecting a subsequent decompression phase of the patient's chest and resuming the delivery of the positive pressure ventilation to the patient based on the detected subsequent decompression phase. It is important to underscore that in each of the multiple embodiments described herein, the tidal volume delivered and the ventilation rate may vary as well. Typically the tidal volume requirements to meet the metabolic needs are about 5-8 ml/kg at a ventilation rate of 6-12 breaths per minute. The split-phase ventilation technique will result in an overall decrease in the ventilation rate to meet the metabolic needs. Ventilation rates with a tidal volume of 5-10 ml/kl may vary between 4-12 breaths/minutes, depending upon the metabolic needs of the patient. It is further understood that a sensor, for example an O2 sensor or a metabolic sensor, may be coupled to the ventilation controller to vary the tidal volume, ventilation rate, and amount of split phase ventilation based upon sensed information from the physiological sensor.

This procedure may be repeated over several cycles of CPR. For example, one cycle may include beginning to compress the chest. As the compression phase begins and is detected (or just prior to the compression phase commencing), all positive pressure breaths to the patient are halted. In some embodiments, an impedance threshold device may also be used during this phase to restrict the inflow of respiratory gases into the lungs. As the chest compression is completed, the compression phase ends and a decompression phase is initiated. This may involve the natural recoil of the chest after the administration of a chest compression and/or may include active decompression force applied by a chest compression device. As the decompression phase is started, a positive pressure breath (or a portion thereof) is delivered to the patient's airway by a ventilation device. As the chest reaches its full recoil position and the decompression phase ends, the positive pressure breath may be ceased, thereby completing one cycle of split-phase CPR. In some embodiments, each positive pressure breath is delivered in a decompression phase of a single cycle, while in other embodiments a single positive pressure breath may be split across the decompression phase of multiple successive cycles. Typically, the positive pressure breaths are delivered solely during successive decompression phases, however in some embodiments a small portion, such as between about 10-30% (commonly about 20%) of each positive pressure breath may be delivered during a compression phase.

In some embodiments, process 500 may be performed in conjunction with HUP CPR techniques, such as those described herein. For example, process 500 may involve elevating the individual's heart, shoulders, and head elevating the individual's heart, shoulders, and head relative to horizontal while repeatedly compressing the patient's chest. The elevation of the patient's upper body may be done at a rate of between about 0.25°/second and about 40°/minute and/or in a controlled manner over a period of time between about 20 seconds and 10 minutes.

In addition or alternatively to one or more of the techniques described above, in some embodiments, the chest compression device will be coupled to a device to elevate the head and thorax that includes one or more support restraints (such as straps, belts, rods, cloth strips, head constraints, and the like) to stabilize the patient on the elevation device.

It will be appreciated that some embodiments may utilize simplified elevation devices. For example, an elevation device may include only a base an upper support, and an actuator for raising and lowering the upper support relative to the base. The upper support may have one or more generally planar surfaces, with one or more of the surfaces optionally being contoured to match a shape of a patient's back. Additionally, the curved profile may make the support surface flexible. This flexibility helps when the elevation device is used in conjunction with a chest compression device, as the flexibility ensures that the right amount force applied to the patient's chest. For example, a central portion of the upper support may flex in the presence of excessive force, thereby acting as a flexible back plate to absorb some of the force. For example, as a plunger of a chest compression device is pressed into the patient's chest, some force is transmitted through the patient to the upper support. The upper support may be configured to bend away from the patient if this transferred force exceeds a threshold. This allows for the delivery of compression at the appropriate depth for patients with differing chest wall sizes and stiffness's. This helps prevent broken ribs and/or other injuries to the patient caused by too much force being applied to the patient's chest, as the flexing back plate, rather than the ribs or other body structures, absorbs a significant portion of the excess force. Such a design is particularly useful when the elevation device is used in conjunction with a chest compression device such as the Lucas device, sold by Physio-Control, Inc. and/or the Zoll AutoPulse. However, it will be appreciated that the flexible upper support may be used in conjunction with any of the embodiments of elevation devices described herein. It should be appreciated that the portion of the elevation device under the heart and thorax could also contain force, pressure, impedance, and/or position sensors to provide feedback to the chest compression device, assuring the proper compression depth and force are delivered, even though the amounts needed to provide the proper CPR may differ from patient to patient and may change over time. In some embodiments, the chest compression device may be coupled with the elevation device 300 in such a manner that compressive and/or decompressive force from the chest compression device remains generally perpendicular (within 5 degrees) to the patient's sternum at all elevation positions.

In some embodiments, the patient's upper body may be elevated at a same angle on a single surface of the upper support, while in other embodiments the upper support may have two or more generally planar surfaces that elevate the heart and head at different angles relative to horizontal. The actuator may be manual and/or automatically driven with operating controls that enable the upper support to be to be raised and lowered in a controlled manner necessary to perform sequential elevation as described herein. For example, the elevation device may be fitted with controllers, motors, threaded rods, lead screws, pneumatic and/or hydraulic actuators, motor driven telescopic rods, other elevation mechanisms, and/or combinations thereof. In some embodiments, the motors may be coupled with a controller or other computing device. The controller may communicate with one or more input devices such as a keypad. This allows a user to select an angle and/or height of the heart and/or head to be raised using the motor and/or other actuator, along with a rate of elevation or other timing element of the elevation process. Additionally, the controller may be coupled with one or more sensors, such as flow and pressure sensors. Sensor inputs may be used to automatically control the motor and angle of the supports based on flow and pressure measurements. A type of CPR and/or intrathoracic pressure regulation may also be controlled using these and/or other sensor inputs. In some embodiments, the electro-mechanical lift mechanisms may include disengagement mechanisms that allow the elevation device to be operated manually. This allows the elevation device to be operable even if a power source for the electromechanical features is unavailable, such as when a battery is dead or when there is no power outlet or other power source available.

In some embodiments, the upper support may define an opening that is configured to receive a portion of a patient's head. This opening may help maintain the patient in the sniffing position for optimal airway management. Oftentimes, a head support may be included on the upper support. It will be appreciated that in some embodiments the head support may extend around the entire opening. The head support may be formed of contoured padding, such as foam padding, such that patients having heads of different sizes and shapes may be supported adequately by the single head support.

In some embodiments, the chest compression device and the elevation device may share a common power source. For example, the chest compression device or the elevation device may include a power source, such as a power cord and/or battery. The non-powered device may then plug into the other device to share the power source. In other embodiments, the chest compression device and the elevation device may be formed as a single device, with the elevation mechanism of the elevation device and the chest compression device both being wired to a single power source. In some embodiments, the power source may also power a ventilator that is built into and/or coupled with the elevation device and/or chest compression device. In some embodiments, such a ventilator may be controlled by the same controller system that operates the elevation device and/or chest compression device.

In some embodiments, the chest compression device and elevation device may be configured to communicate with each other. For example, one or more network or other data cables and/or wireless interfaces may couple processors and/or sensors of each device to one another. Data regarding elevation height, speed of elevation, speed of declination, CPR rate, force applied to the patient, and/or other data may be measured and shared between the devices. Additionally, data from physiological sensors may be shared with the elevation device and/or the chest compression device. This physiological data, such as ICP, blood flow data, blood pressure, intrathoracic pressure measurements, and the like may be used to control the various parameters such as elevation timing, elevation angle, chest compression depth and/or force, and the like.

Additionally, the chest compression device and/or elevation device may be configured to communicate with other devices, such as computers, mobile devices like mobile phones and tablet computers, e-readers, other medical equipment, such as electrocardiographs and defibrillators, and the like. To enable such communication, one or more wired and/or wireless communication networks may be established. For example, various data cables may be used to communicatively couple the chest compression device and/or elevation device to one or more remote devices. In some embodiments, the chest compression device and/or elevation device may include a wireless communications interface that is configured to communicate with one or more remote devices using WiFi, Bluetooth, 3G, 4G, LTE, and/or other wireless communications protocols.

In some embodiments, the elevation device may be coupled with a stretcher-like device for transport that has features that allow the heart and head to be elevated above the plane of the abdomen and lower extremities. For example, the stretcher or stretcher-like device may include rails or other rigid or semi-rigid support members that may be used to secure the elevation device and/or the chest compression device to the stretcher. The elevation device and/or the chest compression device may be coupled to the support members using clamps cables, and/or other securement mechanisms that may ensure the elevation device and/or the chest compression device do not shift relative to the stretcher.

### Example 1

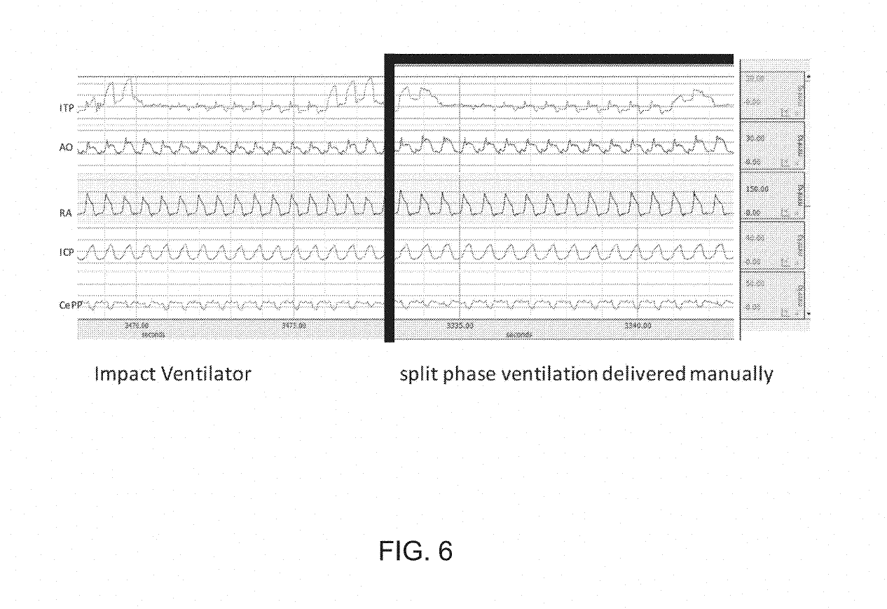

FIG. 6 depicts an example of the physiological effects of split phase ventilation. In this example, a pig received ACD CPR with an impedance threshold device (ITD) which prevents or impedes respiratory gases from flowing to the lungs until a threshold intrathoracic pressure is reached, at which point gases are permitted to freely flow to the lungs. Intrathoracic pressure (ITP), aortic pressure (AO), Right Atrial Pressure (RA), Intracranial pressure (ICP), and the Cerebral Perfusion Pressure (CerPP) were measured. CPR in conjunction with conventional positive pressure ventilation delivered to a pig using an IMPACT mechanical ventilator is compared with manual split phase ventilation with positive pressure ventilations delivered only during the decompression phase. Pressure tracing shows that the peak inspiratory pressures are significantly lower with the split phase ventilation approach and aortic pressure and cerebral perfusion pressures are higher while RA and ICP decompression phase values (min) are lower with the split phase ventilation approach as shown in Table 1 provided below.

TABLE 1

| | ITP | | | | Ao | | | RA | | | ICP | | | CePP | |
|---|---|---|---|---|---|---|---|---|---|---|---|---|---|---|---|
| | max | min | mean | Area | max | min | mean | max | min | mean | max | min | mean | max | mean |
| IMPACT | 36.8 | −8.3 | 4.8 | 73.8 | 35.3 | 13.5 | 21.1 | 142.9 | −8.4 | 44.2 | 31.0 | 4.8 | 15.1 | 13.2 | 6.0 |
| Split Phase | 25.0 | −9.1 | 3.1 | 62.4 | 38.2 | 14.6 | 23.2 | 164.4 | −9.6 | 47.0 | 31.7 | 4.6 | 15.6 | 14.9 | 7.6 |

In some embodiments, a positive pressure delivery device used in accordance with the systems and methods of the present application may be a ventilator that is capable of completely closing off an inspiratory pathway such that a positive pressure ventilation is delivered across multiple decompression cycles, while preventing the delivery of inspiratory airflow during the compression phase of CPR. For example, in some embodiments, the delivery of split-phase ventilations may be done using a ventilator that includes a mechanism that is part of the ventilator to close off the inspiratory pathway completely such that when the pressure within the patient's airway is sub-atmospheric (during the recoil phase of the chest) respiratory gases are prevented from flowing into the patient. This can be accomplished by closing off the inspiratory and expiatory limbs of a ventilator circuit, close to the patient or distant from the patient within the ventilator itself. The operation of ventilators that include such mechanisms to close off inspiratory pathways are further described in in Lurie, Keith G. (1998) “Optimizing Standard Cardiopulmonary Resuscitation With an Inspiratory Impedance Threshold Valve,” CHEST. 1998 April; 113/4:1084-90, the entire contents of which is hereby incorporated by reference.

In addition, while the inspiratory limb of the ventilator circuit can be completely sealed off after a positive pressure breath is delivered respiratory gases can exit the patient through an expiatory limb. The expiratory limb can have no resistance, a set resistance (0-15 cm H2O), and/or potential respiratory gases could be actively withdrawn, such as with a controlled vacuum (e.g. active intrathoracic pressure regulator/ResQVent/VPOD conceptually) throughout the expiratory phase of ventilation. If the ventilator delivers a positive pressure breath and then seals the airway, it functions as an impedance threshold device (ITD). With each chest recoil the intrathoracic pressure is sub-atmospheric. For example, some Siemens® servo ventilators can operate in this manner, thereby eliminating the need for a separate ITD when performing CPR. While such ventilators seal or close the circuit to any inspiration of respiratory gases, the ventilators may or may not allow expiration through a pop off valve that minimizes expiratory resistance.

What is claimed is:

1. A method for ventilating a patient during CPR, the method comprising:
repeatedly compressing the patient's chest resulting in repeating compression and decompression phases of the patient's chest; and
delivering a positive pressure ventilation to the patient during only multiple successive decompression phases of the repeated compressions of the patient's chest.

2. The method for ventilating a patient during CPR of claim 1, further comprising:
sensing one or both of a compression phase and the decompression phase of the repeated compressions of the patient's chest, wherein delivering the positive pressure ventilation is based on the sensing.

3. The method for ventilating a patient during CPR of claim 2, wherein:
sensing one or both of a compression phase and the decompression phase of the repeated compressions of the patient's chest comprises using at least one of a sensor attached to the patient, an airway adjunct attached to the patient, or a compression sensor in a chest compression device that is interfaced with the patient; and
a sensed signal from at least one of the sensor, the airway adjunct, or the compression sensor triggers the positive pressure ventilation during the decompression phase.

4. The method for ventilating a patient during CPR of claim 1, further comprising:
actively decompressing the patient's chest between each compression of the patient's chest.

5. The method for ventilating a patient during CPR of claim 1, further comprising:
elevating the individual's heart, shoulders, and head relative to horizontal while repeatedly compressing the patient's chest.

6. The method for ventilating a patient during CPR of claim 5, wherein:
elevating the individual's heart, shoulders, and head is performed at a rate of between about 0.25°/second and about 40°/minute.

7. A method for ventilating a patient during CPR, the method comprising:
repeatedly compressing the patient's chest resulting in repeating compression and decompression phases of the patient's chest;
detecting a decompression phase of the patient's chest; and
initiating delivery of a positive pressure ventilation to the patient based on the detected decompression phase, wherein the positive pressure ventilation is delivered at least primarily during the detected decompression phase.

8. The method for ventilating a patient during CPR of claim 6, further comprising:
detecting a compression phase of the patient's chest; and
ceasing the delivery of the positive pressure ventilation upon the detection of the compression phase.

9. The method for ventilating a patient during CPR of claim 7, further comprising:
detecting a subsequent decompression phase of the patient's chest; and
resuming the delivery of the positive pressure ventilation to the patient based on the detected subsequent decompression phase.

10. The method for ventilating a patient during CPR of claim 6, further comprising:
elevating the individual's heart, shoulders, and head relative to horizontal while repeatedly compressing the patient's chest.

11. The method for ventilating a patient during CPR of claim 9, wherein:
the individual's heart, shoulders, and head are elevated in a controlled manner over a period of time between about 20 seconds and 10 minutes.

12. The method for ventilating a patient during CPR of claim 2, wherein:
the positive pressure ventilation is delivered to the patient only during the multiple successive decompression phases.

13. A system for delivering split-phase positive pressure ventilations for use in cardiopulmonary resuscitation (CPR), comprising:
a positive pressure delivery device;
at least one sensor that is configured to:
be coupled to one or both of a patient or a device that interacts with a patient during CPR; and
detect a decompression phase of CPR; and
a controller system that is coupled with the positive pressure delivery device and in communication with the at least one sensor, the controller system being configured to cause the positive pressure delivery device to deliver positive pressure ventilation primarily during the decompression phase of CPR over at least two decompression cycles.

14. The system for delivering split-phase positive pressure ventilations for use in cardiopulmonary resuscitation (CPR), of claim 13, wherein:
the at least one sensor is further configured to detect a compression phase of the patient's chest; and
the controller system is further configured to cease the delivery of the positive pressure ventilation base on the detection of the compression phase.

15. The system for delivering split-phase positive pressure ventilations for use in cardiopulmonary resuscitation (CPR), of claim 13, wherein:
the at least one sensor comprises an intrathoracic pressure sensor that is configured to detect the compression phase when an intrathoracic pressure of the individual exceeds a predetermined threshold.

16. The system for delivering split-phase positive pressure ventilations for use in cardiopulmonary resuscitation (CPR), of claim 15, wherein:
the predetermined threshold is about 10 mmHg.

17. The system for delivering split-phase positive pressure ventilations for use in cardiopulmonary resuscitation (CPR), of claim 13, further comprising:
an elevation device comprising:
a base;
an upper support coupled with the base, wherein the upper support is configured to elevate an individual's heart, shoulders, and head relative to horizontal, the upper support being moveable between a first elevated position and a second elevated position, the second elevated position having a level of elevation relative to the horizontal which is greater than that of the first elevated position;
an adjustment mechanism coupled with the upper support that is configured to adjust a level of elevation of the upper support; and
a chest compression device for performing chest compressions coupled with one or both of the base and the upper support.

18. The system for delivering split-phase positive pressure ventilations for use in cardiopulmonary resuscitation (CPR), of claim 17, wherein:
the chest compression device is further configured to actively decompress the individual's chest between each chest compression.

19. The system for delivering split-phase positive pressure ventilations for use in cardiopulmonary resuscitation (CPR), of claim 17, wherein:
the elevation device further comprises an additional controller system that is coupled with the adjustment mechanism and the chest compression device, the additional controller system being configured to:
actuate the chest compression device for a period of time with the upper support in the first elevated position;
subsequently move the upper support from the first elevated position to the second elevated position whilst the chest compression device remains actuated; and
actuate the chest compression device with the upper support in the second elevated position.

20. The system for delivering split-phase positive pressure ventilations for use in cardiopulmonary resuscitation (CPR), of claim 13, wherein:
the controller system and the additional controller system are the same.
